(19) United States
(12) Patent Application Publication
(10) Pub. No.: US 2014/0341940 A1
(43) Pub. Date: Nov. 20, 2014

(54) IMMUNOMODULATOR FOR THE TREATMENT OF CANCEROUS TUMORS IN THE EPITHELIAL TISSUE LINING SURFACES INSIDE OR OUTSIDE BODY ORGANS

(71) Applicant: Iseu da Silva NUNES, Campinas (BR)

(72) Inventor: Iseu da Silva NUNES, Campinas (BR)

(21) Appl. No.: 14/444,436

(22) Filed: Jul. 28, 2014

Related U.S. Application Data
(63) Continuation-in-part of application No. 14/357,490.

(51) Int. Cl. A61K 9/00; A61K 31/704; A61K 31/407; A61K 39/00; A61K 39/04

(52) U.S. Cl. CPC A61K 9/0014; A61K 39/0011; A61K 39/04; A61K 31/407; A61K 31/704; A61K 2039/54; A61K 2039/585
USPC 424/185.1

(57) ABSTRACT

The present invention provides a new treatment for noninvasive cancers in the epithelial tissue lining inside and outside surfaces, including the topical use of the immunomodulator, either alone or in association or combination with other drug and non-drug treatments.

The present patent application concerns the use of an immunomodulator for the treatment of cancerous tumors in the epithelial tissue lining surfaces outside and inside the body, which can be used alone, or in combination or association with other therapies.

### Epithelial Tissue—General Description

The epithelial tissue is a special kind of cell structure that covers the entire outer surface of the body or skin, and all internal body cavities, such as the pleural, pericardial and peritoneal cavities. It also forms the lining of the heart, blood and lymphatic vessels, digestive tract and genitourinary tract.

Consists almost exclusively of juxtaposed cells, with very little of no intercellular substance between them. The cells adhere to each other by means of intercellular junctions or by proteins on the cell membrane.

The lining epithelial tissue does not have its own vascular system, and, thus, nutrients for the cells are obtained from capillaries in adjacent connective tissue via diffusion.

The epithelial tissue performs many important functions, such as: protection and covering (e.g. skin), secretion (e.g. stomach), mixed functions of secretion and absorption (e.g. intestine), waterproofing (e.g. urinary bladder).

### Epithelial Tissue and Cancer

Finally, it should be noted that, just as in any other tissue, malignant tumors can originate in the epithelial tissue, and it is noteworthy that many malignant tumors in humans and animals originate or are located in this particular type of tissue.

This fact is not surprising, considering that the epithelial tissue, due to its characteristics and functions, has cells that must be constantly renewed and are almost continuously exposed to aggressive external agents such as solar radiations and potentially carcinogenic chemical compounds.

### Routes of Drug Administration

The route of administration of medicines and compounds to subjects in need thereof is determined primarily by the properties of the drug to be used (for instance, water or lipid solubility, etc.) and also by the therapeutic objectives (for instance, the desirability of a rapid onset of action or restriction to a local site. The invention, as explained in the report acts locally, that is, its therapeutic action is directed against a localized lesion or disease.

### Epithelial Tissue and Types of Treatment—State of the Art

It should also be noted that the epithelial tissue does not have an efficient blood supply or even does not have vascular tissue which hinders or prevents the use of drugs or medications that depend on transportation routes, such as the circulatory or lymphatic system, to reach body tissues of this particular region.

This is relevant for the treatment of malignant lesions located in the most superficial layer of the lining epithelium, once the use of therapies containing agents of systemic action is not very effective, or is virtually useless for the treatment of malignant or pre-malignant lesions in the superficial layer of the epithelial tissue lining surfaces outside or inside body organs.

Therefore, in the closest state of the art, the medical treatments used include special techniques and procedures, such as surgical procedures, radiotherapy and drugs with local or topical effect, which work more effectively in the epithelial tissue lining surfaces outside or inside the body.

The main goal of the topical treatment of tumors in this specific region is to eradicate tumors of the epithelial tissue lining surfaces outside or inside body organs before they become invasive and spread to adjacent tissues and then enter the body.

For the purposes of the present invention and to facilitate understanding of the state of the art it is important to mention that lining epithelial tissue can be accessed for therapeutic purposes, that is, for performing local excisions and topical treatments inside or outside the body.

This occurs, for example, in the case of skin tumors locally treated by surgical removal, radiation therapy, phototherapy and chemotherapy, which may be used alone or in combination. Likewise, epithelial tumors of the inner surface of an organ (e.g. urinary bladder) can be reached and topically treated by means of surgical procedures, various types of radiations and also drugs with topical action, after deposition in the regions of interest.

In the particular case of medicines, some special techniques and equipment can be used in the region of interest long enough to exert topical action in the affected tissue.

As an example, the treatment of tumors of the inner surface of the urinary bladder epithelium, treated by irrigation or contact with chemotherapy drugs, which for these purposes are introduced into the bladder with the use of catheters for insertion into the bladder.

Finally, it is well-known in the closest state of the art that there are other types of non-drug therapies available to treat tumors that affect the lining epithelial tissue, e.g., surgical treatments and radiation therapy, which can also be used in combination or association with drugs to maximize the therapeutic response.

### Safety Requirements for the Development of Therapies for Use in the Epithelial System

Medications or procedures to be used for the treatment of malignant lesions in the external or internal surfaces of the epithelial tissue should have topical action, restricted to the epithelium, to prevent them from reaching blood vessels and other transport systems of the body, causing toxicity to other organs.

As a general rule, it can be said that any procedure or medication developed for the topical treatment of malignant lesions of the epithelial tissue covering internal surfaces of the body can be used for the treatment of malignant lesions and tumors located in the epithelial tissue lining surfaces outside body organs.

The opposite is not always possible, that is, some drugs and therapies developed for use in epithelial cells that line external surfaces of the body are not suitable for use in tumors located in the epithelium of internal surfaces because of high toxicity problems.

The reason for this is that, besides being more sensitive to aggressive agents, the epithelium that covers internal surfaces of the body is more sensitive to aggressive agents, and sometimes has a higher degree of permeability, compared to the epithelium that covers external body surfaces.

Differences in the degree of permeability between the epithelium of the internal and external surfaces of the body are directly related to the function performed by the organ covered by these tissues.

This is observed, for example, in the case of the epithelial cells that line the intestine, which can carry substances into the body and therefore can more easily absorb substances than the skin, which performs functions related to protection against penetration of external agents into the body and of elimination of substances from the body, as in the case of sweat.

In practical terms, this prevents products with high systemic toxicity, which can be used for therapeutic purposes in the skin because of its relatively low absorption capacity, from being used to treat diseases in the intestinal region, due to the high absorption capacity of the lining epithelium of this anatomical region.

As a practical example of a procedure used for the two types of epithelial surface, we can cite the treatment of cancerous lesions of the stomach lining, through the use of pulses of high energy light (laser), a procedure that is also widely used to eradicate malignant lesions in the skin.

Treatments based on corrosive chemical agents, such as those used to remove warts and malignant or premalignant lesions in the outer layer of skin (epidermis) cannot be used in the treatment of lesions in the epithelial tissue of internal surfaces due to their potential for indiscriminate damage to healthy tissues, and if these agents are absorbed by the body they can cause serious or fatal intoxication.

In the state of the art, we can cite Podophylin, a compound derived from a plant of the species Podophyllum Emodi, used for the removal of superficial skin lesions, including warts usually caused by HPV and also malignant and premalignant lesions. However, it cannot be used in the epithelial tissue lining surfaces inside body organs due to its toxic properties and because it can be absorbed into the body.

The drug imiquimod (IUPAC: 1-(2-methylpropyl)-1H-imidazo[4,5-c]quinolin-4-amine, C₁₄H₁₆N₄), an immunomodulator or local immune response modifier, which has high toxicity to various cell structures, can be cited as another example.

Consequently, in clinical practice, this compound is only allowed for the topical treatment of premalignant lesions caused by HPV virus, such as condyloma acuminata, or for skin papilloma, or else for the treatment of basal cell carcinoma in the epithelium of the skin surface.

Due to this toxicity, imiquimod cannot be used in the treatment of malignant lesions in the epithelium of internal surfaces of the body, such as, premalignant or malignant lesions in the intestine, uterus, stomach and urinary bladder, because it can be absorbed by the mucosa of these organs.

Finally, it should be noted that the main purpose of the treatment of malignant or premalignant lesions in the lining epithelium of internal or external surfaces of the body is the elimination of such lesions before they propagate into deeper layers, which unlike the surface layers are well irrigated, favoring the dissemination of cancer to other organs and systems of the body.

When the lesion extends beyond the surface layer of the lining epithelium and reaches the basal layer or further, the treatment with drugs with predominantly topical (local) action is not effective, since many compounds are only effective or can be used locally due to toxicity or permeability problems.

Since the treatment of malignant lesions that extend beyond the surface layer of the lining epithelium is complicated because of the above mentioned reasons, other types of therapies should be used, or else the use of chemotherapy drugs with systemic effect, which are usually toxic to the most of patients.

For example, local (intravesical) immunotherapy with BCG vaccine used in the topical treatment for cancerous lesions in the epithelial lining of the urinary bladder is ineffective to treat infiltrating tumors, that is, malignant tumors extending beyond the surface layer and invading the basal or muscle layer of the bladder, as it will be detailed in the present report. Furthermore, in the state of art, for treatment of urinary bladder cancer, due its high toxicity and the possibility of systemic infection of the patients, BCG vaccine is mainly indicated for intravesical use.

Therefore, the topical therapy in the closest state of the art, which includes most current therapeutic agents cited here in the case of urinary bladder cancer that infiltrates to the muscle layer cited here, for example, is ineffective, and consequently radical surgery for removal of the bladder, a procedure called cystectomy, followed or not by radiotherapy and systemic chemotherapy, is required.

In the state of the art, cystectomy, or partial or total removal of the urinary bladder, is the procedure indicated for the treatment of invasive tumors, and even for superficial bladder tumors that do not respond to standard therapy, which consists in local surgical excision of the tumor followed by immunotherapy or topical chemotherapy, as will be explained in detail herein.

On the one hand, this surgical procedure (cystectomy) can prevent further spread of cancer to new areas, but on the other hand, it causes significant loss of quality of life for most patients. For reference: Clark, Peter E. et al—Radical cystectomy in the elderly—Comparison of clinical outcomes between younger and older patients. Cancer-Volume 104, Issue 1, pages 36-43, 1 Jul. 2005. See also: Ganesh S P et al. Assessment of perioperative psychological distress in patients undergoing radical cystectomy for bladder cancer. The Journal of Urology, Volume 172, Issue 5, Part 1, November 2004, Pages 1814-1817

### The Need for Improvements in the State of the Art

The above examples clearly show that, due to the considerable shortcomings of the available treatments and drugs, new types of treatment for cancer originated or located in the epithelial lining of internal and external surfaces of the body are needed in the state of the art, to replace or complement the current treatments, maximizing their efficiency.

It would be highly desirable that these new therapeutic modalities had no unwanted side effects commonly observed in the treatments available in the state of the art or reduced toxicity to end users.

The creative or inventive activity should therefore be aimed to the discovery of new therapeutic modalities, including new methods of treatment and compounds, used alone or in association, and which are comparatively more beneficial than the current treatments in terms of effectiveness and safety.

If these therapeutic modalities, because of their characteristics or properties, can be used in the topical treatment of malignant tumors in the lining epithelium of internal and external surfaces of the body, without toxicity and contributing to reduce the toxicity of the current treatments, this would undoubtedly constitute a considerable improvement in the state of the art.

The present invention satisfies all these needs, by providing an immunomodulator, which, in an innovative way, allows the treatment of malignant tumors located in the epithelial lining of internal and external surfaces of the body, and does not cause significant toxicity that disallows its use, unlike most compounds available in the state of the art. And in order to demonstrate the usefulness, inventive level of activity involved and comparative advantages of the invention reported here, several practical examples of its use in malignant lesions in the epithelial lining of internal and external surfaces of the body in experimental animals are presented.

The results are presented only for illustrative purposes to help understand the novelty, usefulness and advantages of this invention, and by no means intend to limit the scope or the field of application of the present invention.

### Cancer in the Epithelial Tissue Lining Surfaces Inside Body Organs—Cancer of the Urinary Bladder—State of the Art

Urinary bladder cancer is one of the most important cancers that affect or originate in the epithelial lining of inside surfaces.

Bladder cancer is the most common tumor of the urinary tract after prostate cancer, and one of the most common cancers, with a male to female ratio of 4:1 in the United States. For reference: American Cancer Society. Cancer Facts and Figures 2009. Atlanta, Ga.: American Cancer Society; 2009. http://www.cancer.org. Accessed Jun. 4, 2010.).

From an economic point of view, patients with bladder cancer represent the highest annual costs for the treatment of all types of cancer in the United States, according to reports from the health sector for this specific disease. For reference: Simons M P et al. Role of neutrophils in BCG immunotherapy for bladder cancer. Urol Oncol 2008; 26:341-5, PubMed: 18593617)

### Tumor Standardized Classification System—State of the Art—the TNM System (TNM Staging System)

The TNM system is one of the most widely used cancer staging systems. This system has been accepted by the Union for International Cancer Control (UICC) and the American Joint Committee on Cancer (AJCC).

### TNM System—Classification of Urinary Bladder Cancers

According to the TNM system, tumors in the urinary bladder can be classified as follows (Table 1).

TABLE 1
Classification of urinary bladder cancers using the TMN system

| TX | Primary tumor cannot be assessed |
|---|---|
| T0 | No evidence of primary tumor |
| Ta | Noninvasive papillary carcinoma |
| Tis | Carcinoma in situ: “flat tumor” |
| T1 | Tumor invades subepithelial connective tissue |
| T2 | Tumor invades muscle |
| | T2a - Tumor invades superficial muscle (inner half) |
| | T2b - Tumor invades deep muscle (outer half) |
| T3 | Tumor invades perivesical tissue |
| | T3a - microscopically |
| | T3b - macroscopically (extra vesical mass) |
| T4 | Tumor invades any of the following structures: prostate, |
| | uterus, vagina, pelvic wall or abdominal wall. |
| | T4a - Tumor invades prostate, uterus or vagina. |
| | T4b - Tumor invades pelvic wall or abdominal wall |

Approximately 75% of tumors located in the epithelial lining of the urinary bladder detected at the time of diagnosis are classified as non-muscle invasive bladder cancers, or else classified according to the TNM system as Ta, T is, T1 (Table 1).

These tumors classified as Ta, T1, T is, are confined to the most superficial mucosa of the urinary bladder or in the lamina propria.

The remaining 25% of all the types, at the time of diagnosis, are classified as muscle invasive cancers, or else, into the groups T2, T3, T4 of the TNM System (Table 1). For reference: (Sullivan P S et al. Urine cytology and adjunct markers for detection and surveillance of bladder cancer. Am J Transl Res 2010; 2(4):412-440)

### Treatment Strategies

For the purpose of preparation of therapeutic strategies, urinary bladder tumors are usually divided into: superficial and invasive, with superficial tumors including those classified into stages T is, Ta and T1 by the TNM system (Table 1). According to this system, penetration of muscle layer by the tumor mass identifies invasive bladder tumors, that is, T2, T3 and T4 (Table 1).

### Treatment

The initial approach for all bladder tumors is a surgical procedure called Transurethral Resection of Bladder Tumors or TURBT.

The so-called Transurethral Resection of Bladder Tumors (TURBT) is the standard procedure for diagnosis, staging and initial treatment of tumors located in the epithelial lining of the urinary bladder.

Briefly, transurethral resection of bladder tumors or TURBT comprises the surgical removal of tumors or lesions affecting the urinary bladder suspected to be malignant. Such tumors or lesions are removed (resected) with the aid of surgical handles, laser scalpels and/or electrocauterization

The surgical technique called Transurethral Resection of Bladder Tumors (TURBT) is well-known in the state of the art, and more detailed information can be obtained e.g. in EAU guidelines on the treatment of urothelial carcinoma, a non-muscle invasive bladder cancer. For reference: Babjuk et al. EAU Guidelines on Non-Muscle-Invasive Urothelial Carcinoma of the Bladder. Eur Urol 2008 August; 54(2): 303-14.

Subsequently, and at the end of the procedure (TURBT), tissue fragments are removed from the region by aspiration, for conducting anatomopathological examination and histological assessment of the lesions found, for purposes of classification of the tumor, usually according to the TNM system, aiming to establish the need for and/or type of treatment patient will require.

As already mentioned, after completion of the TURBT, the tissue fragments removed are examined by several techniques, such as histological analysis of tissues to allow tumor classification using the TNM system, to establish the need for and/or type of treatment the patient will subsequently require.

As shall be explained in more detail in this report, for epithelial tumors classified as high grade by the TNM system (Ta, T1, Tis), the best procedure available in the state of the art is a local excision of the tumor (TURBT) for removal and staging of tumors, followed by adjuvant intravesical therapy using chemotherapy or immunotherapy compounds.

In the state of the art, the best treatment for tumors originated in or affecting the epithelial tissue lining surfaces inside or outside the body uses a combination of treatments involving the removal of lesions located by surgical procedures, followed by adjuvant therapy using drugs or compounds of topical action.

The main purposes of this postsurgical treatment in the case of tumors affecting the epithelial tissue lining inside or outside body surfaces, and in the particular case of bladder cancer, is the elimination of tumors that might have gone undetected during the surgical stage, and also prevent tumor recurrence.

### Treatments Available in the State of the Art

Although any malignant tumor, including those in the epithelial region, can spread throughout the body, in the specific case of patients with tumor in the superficial epithelium of the urinary bladder, those classified as high degree Ta, T1, T is (Table 1) have the highest risk of recurrence and progression, or else, of extending beyond the epithelium, becoming invasive and spreading throughout the body, if treated by transurethral resection only, or else, by local excision.

Therefore, after surgical removal of the lesions (TURBT) in the epithelial lining of the internal surface of the organ, the best treatment available in the state of the art, to avoid progression or tumor recurrence is the treatment of the urinary bladder epithelium using chemotherapy and immunotherapy compounds.

In the specific case of urinary bladder cancer, the topical treatment of the vesical epithelium using chemotherapy or immunotherapy compounds is called intravesical therapy or adjuvant intravesical therapy.

The so-called intravesical therapy is a modality of topical treatment that, in the specific case of the epithelium or epithelial surface of the bladder, basically consists in the introduction of agents with anticancer properties in the bladder, through appropriate means, where they will remain for a certain period of time in contact with the affected epithelium.

The main purposes of the topical intravesical treatment are: to minimize the possibility of persistence and tumor relapse, inhibit the formation of new tumors and ultimately attempt to avoid or slow the progression of superficial lesions located in the epithelium for other tissues or organs.

For illustrative purposes only, some examples of therapeutic agents in the state of the art used in the treatment of malignant lesions in the epithelium of the urinary bladder are shown in Table 2.

According to their mode of action, these agents can be generally classified in two groups or classes: chemotherapy and immunotherapy drugs. The chemotherapy drugs comprise all compounds capable of destructive action against tumor cells, while those classified as immunotherapy drugs comprise compounds that act indirectly via the patient's immune system, which, in turn, stimulated by these drugs, is able to eliminate or slow the progress of the cancer.

TABLE 2
CHEMOTHERAPY AND IMMUNOTHERAPY DRUGS
MOST USED FOR INTRAVESICAL APPLICATION IN
BLADDER CANCER

| | POSSIBLE |
|---|---|
| | MECHANISMS OF ACTION |
| CHEMOTHERAPY DRUGS | |
| Mitomycin C | Antibiotic, inhibition of DNA synthesis. |
| Thiotepa | Alkylating agent |
| Doxorubicin | Inhibition of topoisomerase, inhibition |
| | of DNA synthesis. |
| Gemcitabine | Inhibition of DNA and RNA synthesis. |
| Valrubicin | Inhibition of topoisomerase, inhibition |
| | of DNA synthesis. |
| IMMUNOTHERAPY DRUGS | |
| Bacillus Calmette-Guérin | Stimulation of the immune system |
| (BCG vaccine) | |
| Interferon (IFN) | Stimulation of the immune system, |
| | lymphocyte activation, antiangiogenic, |
| | antiproliferative. |
| Interleukin-2 (IL-2) | Stimulation of the immune system |

### Chemotherapy and Immunotherapy in Bladder Cancer—Main Therapeutic Agents

As previously mentioned, the treatment of malignant lesions in the lining epithelium involves the combination of different therapies, beginning with a surgery to remove the tumors followed by treatment of the affected region with the use of chemotherapy and/or immunotherapy to eradicate residual tumors and prevent disease recurrence.

In the closest state of the art, in the case of postsurgical treatment of malignant lesions in the epithelium of the urinary bladder, intravesical immunotherapy using agents capable of stimulating the immune system is considered the most efficient therapeutic procedure compared to the use of chemotherapeutic compounds, regarding the prevention of cancer recurrence. For reference: Hall M C, Chang S S, Dalbagni G, et al. Guideline for the management of nonmuscle invasive bladder cancer (stages Ta, T1, and Tis): 2007 update. J Urol. 2007; 178(6):2314-2330.

Among the agents that may be used topically for the treatment of epithelial cancer of the urinary bladder, the most effective immunotherapeutic agent used in the closest state of art is BCG vaccine, which was originally developed for the prophylactic treatment of tuberculosis.

The use of BCG vaccine in the postsurgical treatment of cancerous lesions in the epithelial lining of the urinary bladder began in the 70s with the pioneering work of Morales and collaborators, who developed a new and innovative practical use for a product that was previously used only for prophylaxis of tuberculosis. For reference: Alexandroff A B et al. BCG immunotherapy of bladder cancer: 20 years on. The Lancet, Volume 353, Issue 9165, Pages 1689-1694, 15 May 1999.

Since then, it has become the leading postsurgical treatment used in the state of the art as adjuvant therapy in the treatment of epithelial cancer, especially epithelial tumor of the urinary bladder. For reference: Alexandroff A B et al. BCG immunotherapy of bladder cancer: 20 years on. The Lancet, Volume 353, Issue 9165, Pages 1689-1694, 15 May 1999.

### Classification of the Degree of Risk of the Lesions and Intravesical Adjuvant Therapy

Although the treatment of any epithelial lesion in the urinary bladder considered cancerous or with the potential to develop into cancer begins with the surgical removal of the tumor (TURBT), the subsequent treatment may vary, depending on the degree of classification of the cancer.

For tumors classified as having high degree of recurrence, though low risk of progression, the most commonly used criteria for treatment in the state of the art indicate the need for intravesical adjuvant therapy following surgical resection of lesions (TURBT), using intravesical agents.

Therefore, the recommendations of the AUA (American urological association) include the use of adjuvant therapy with chemotherapeutic agents, such as Mitomycin C or immunotherapeutic agents, such as BCG vaccine. For reference: Hall M C, Chang S S, Dalbagni G, et al. Guideline for the management of nonmuscle invasive bladder cancer (stages Ta, T1, and Tis): 2007 update. J Urol. 2007; 178(6):2314-2330

For patients with tumors classified as having high risk of recurrence and also high risk of progression (Ta, T1, Tis), the most common recommendation in the state of the art after surgical removal of the tumors includes an initial phase of therapy where only BCG vaccine induction is used followed by BCG maintenance therapy.

For reference, we cite: Hall M C, Chang S S, Dalbagni G, et al. Guideline for the management of nonmuscle invasive bladder cancer (stages Ta, T1, and Tis): 2007 update. J Urol. 2007; 178(6):2314-2330.

In the state of the art, according to the results obtained in several clinical trials, the use of intravesical therapy with BCG vaccine indicated that this immunotherapeutic drug when compared with chemotherapeutic agents such as Mitomycin C was clearly more effective. For reference: Sylvester R J et al—Bacillus Calmette-Guerin versus Chemotherapy for the intravesical treatment of patients with carcinoma in situ of the bladder: A meta-analysis of the published results of randomized clinical trials. J Urol. 2005 July; 174(1):86-91; discussion 91-2.

For example, the results published by the American Urological Association (AUA) show a recurrence rate of 34%, after 5 years, for patients treated with BCG induction therapy, followed by BCG maintenance therapy, compared to a recurrence rate of 64% for patients treated with chemotherapy drugs (Mitomycin C). For reference: Sylvester R J et al—Bacillus Calmette-Guerin versus Chemotherapy for the intravesical treatment of patients with carcinoma in situ of the bladder: A meta-analysis of the published results of randomized clinical trials. J Urol. 2005 July; 174(1):86-91; discussion 91-2.

### Intravesical Maintenance Immunotherapy

Maintenance therapy is defined in the state of the art as the periodical exposure of the epithelial tissue (urothelium) for considerable periods of time to an intravesical antineoplastic agent such as Mitomycin C or BCG.

The use of maintenance therapy has proved to be an important strategy for reducing cancer recurrence, as well as disease progression to more advanced and aggressive stages.

The use of BCG maintenance therapy, after transurethral resection of bladder tumors (TURBT), is considered the most effective treatment, in the state of the art, in reducing the rate of tumor recurrence compared to other intravesical agents.

The most relevant reference in the state of the art of effectiveness of maintenance therapy using BCG vaccine is the clinical study called SWOG 8507 conducted by the Southwest Oncology Group or SWOG.

In the abovementioned study, that is, the SWOG 8507, patients with epithelial cancer at high risk or progression were treated with BCG maintenance therapy after resection surgery (TURBT) every 6 months, compared to patients treated only with surgery and who did not receive BCG therapy.

The maintenance treatment lasted for 3 years. All patients were followed for 5 years after completion of their treatments for assessment of disease recurrence. Only 16% of the patients in the SWOG 8507 study completed the entire period of intended use of the treatment due to problems related to toxicity of BCG.

However, the rate of recurrence after 5 years was 60% for the patients treated with BCG maintenance therapy, compared to 41% of non-recurrence, in the same period of time, for the patients not treated with the referred therapy.

For references and results of the SWOG 8507 study: Lamm D L, Blumenstein B A, Crissman J D, et al. Maintenance Bacillus Calmette-Guérin immunotherapy for recurrent TA, T1 and carcinoma in situ transitional cell carcinoma of the bladder: a randomized Southwest Oncology Group Study. J Urol. 2000; 163(4): 1124-1129.

After the completion of this study, the use of BCG vaccine following transurethral resection of bladder tumors (TURBT), has definitely established itself in the state of the art as the best treatment option for malignant tumors at high risk of progression affecting the lining epithelium of the urinary bladder.

Complications and Main Adverse Effects of Intravesical Therapy with Drugs

Table 3 relates the most common adverse side effects associated with the use of several compounds used in intravesical therapy. The symptoms and effects related to the use of BCG are the most frequently cited in the literature available in the state of the art, reflecting the fact that it is the most frequently used adjuvant therapy (Table 3).

TABLE 3
Adverse side effects and complications of the
drugs used in the intravesical therapy for bladder cancer

| | | (%) patients |
|---|---|---|
| Drug | Adverse side effects | affected |
| Bacillus Calmette-Guérin | Cystitis | >50 |
| (BCG) | Fever (low grade) | 25 |
| | Prostatitis | <5 |
| | Epididymitis | <2 |
| | Fever (high grade) | 3 |
| | Septicemia | <0.5 |
| | Generalized infection | — |
| | | Rarely |
| Mitomycin C | Hematuria | 10 |
| | Myelosuppression | <10 |
| | Skin rash | <10 |
| | Bladder contraction | <2 |
| | Tissue necrosis | Rarely |
| Thiotepa | Chemical cystitis | 20 |
| | Myelosuppression | 10 |
| Doxorubicin | Chemical cystitis | 25 |
| | Allergic reaction | 1 |

Bibliographic references (Table 3): Hall M C, Chang S S, Dalbagni G, et al. Guideline for the management of nonmuscle invasive bladder cancer (stages Ta, T1, and Tis): 2007 update. J Urol. 2007; 178(6):2314-2330

Adjuvant Immunotherapy with BCG—Practical Problems and Contraindications

Although the BCG vaccine used as therapeutic adjuvant in the topical treatment of bladder cancer is the most effective therapy available in the state of the art to prevent tumor recurrence and progression after transurethral resection of bladder cancer, a large number of patients experience recurrence and progression of the disease, despite undergoing the referred treatment.

The treatment with BCG therapy is ineffective in around 30 to 40% of the patients, and even among those patients who respond well to treatment in the early days, 30% will suffer a relapse, that is, recurrence of bladder epithelium cancer after a certain period of use of BCG therapy. For reference: Palou Redorta J. Management of BCG Failures, Eur Urol. (2006; 49:779-80)

Additionally, the application of BCG vaccine in intravesical treatment of bladder cancer is considerably hindered in practice by the occurrence of unwanted side effects, which are detected in around 90% of the patients, consisting of symptoms of irritation, presence of blood in urine (haematuria), dysuria and emergency, leading to early discontinuation of treatment in many cases. For references: Perabo F G, Willert P L, Wirger A et al (2005) Preclinical evaluation of superantigen (Staphylococcal Enterotoxin B) in the intravesical immunotherapy of superficial bladder cancer. Int J Cancer 115:591-598 and Askeland et al. Bladder Cancer Immunotherapy: BCG and Beyond. Advances in Urology. Volume 2012 (2012), Article ID 181987. http://dx.doi.org/10.1155/2012/181987

In a clinical study conducted in 2000, in the United States, by the Southwest Oncology Group (SWOG 85071), only 16% of the total number of patients who received intravesical BCG to treat bladder cancer completed the treatment because of unwanted side effects. For reference: Lamm D L, Blumenstein B A, Crissman J D, et al. Maintenance Bacillus Calmette-Guérin immunotherapy for recurrent TA, T1 and carcinoma in situ transitional cell carcinoma of the bladder: a randomized Southwest Oncology Group Study. J Urol. 2000; 163(4): 1124-1129.

Additionally, because of episodes of infection, including systemic infection by a microorganism (Mycobacterium bovis) used in the preparation of BCG vaccine, doses need to be reduced, prolonged and expensive treatment with antibiotics are required and finally in some cases, interruption of BCG therapy. For reference: Lamm D L, Blumenstein B A, Crissman J D, et al. Maintenance bacillus Calmette-Guerin immunotherapy for recurrent TA, T1 and carcinoma in situ transitional cell carcinoma of the bladder: a randomized Southwest Oncology Group Study. J Urol 2000; 163:1124-9. See also: Palou Redorta J. e Management of BCG “Failures”. Eur Urol. (2006; 49:779-80).

Finally, according to recommendations of the American Urolological Association (AUA), the BCG vaccine is not recommended for use in intravesical treatment of tumors in patients under the following conditions: a) in the two weeks following bladder surgery (TURBT), b) shortly after traumatic bladder catheterization, c) in the presence of hematuria, d) in pregnant or breast feeding patients, e) in immunosuppressed patients or in patients taking drugs with immunosuppressive effects, f) in patients under radiotherapy, g) in patients with urinary tract infection and with a history of allergic reactions or who had previous reactions to BCG. For reference: Hall M C et al. Guideline for the Management of Nonmuscle Invasive Bladder Cancer: (Stages Ta, T1, and Tis): Update (2007). J Urol. 2007 December; 178(6):2314-30.

Non-Surgical Options for the Case of Failure of Immunotherapy with BCG—State of the Art

As set forth, in the closest state of the art, the best therapy available to treat urinary bladder cancer, which is BCG vaccine, has important limitations because of the high rate of cancer recurrence and the high incidence of unwanted side effects, leading many patients to discontinue treatment.

The high incidence of episodes of toxicity associated to the use of BCG vaccine, certainly prevents its wide use as intravesical therapy for the treatment of all urinary bladder cancers following transurethral resection (TURBT), except for the treatment of the most aggressive cancers that affect this region, which would be considered only for the treatment of the most aggressive tumors that affect this region.

In the case of failure of BCG vaccine therapy, which includes both tumor recurrence and discontinuation of treatment and even abandonment of treatment after episode of severe toxicity for the user, without tumor removal, the most indicated treatment in the state of the art is total or partial removal of the urinary bladder, which is called cystectomy.

However, as cited above, this is a high risk surgery that invariably results in significant worsening of the quality of life of patients, and, thus, it should be avoided or delayed as much as possible.

In the closest state of the art, one of the possible options of treatment for the case of failure of intravesical therapy with BCG, aiming to avoid or delay total bladder removal (cystectomy) is its replacement in the intravesical treatment of the lining epithelium of the urinary bladder by chemotherapeutic agents such as Mitomycin C, Thiotepa, Adriamycin, Valrubicin and some others.

Although the use of agents such as chemotherapy drugs can be considered a valid option for treating epithelial tumors of the urinary bladder classified as low grade, this does not apply to patients with high-grade cancer, i.e., classified according to the TNM systems in grades Ta, T1, Tis.

For example, in a study conducted in 1999, only 19% of patients with bladder cancer in use of intravesical chemotherapy (Mitomycin C), had no recurrence of cancer during the follow-up period, compared with 39% of the patients in use of BCG that are disease free. For reference: (Malmstrom P U, Wijkstrom H, Lundholm C, et al. 5-year follow-up of a randomized prospective study comparing mitomycin C and bacillus Calmette-Guerin in patients with superficial bladder carcinoma. J Urol 1999; 161:1124-7.

In short, the use of intravesical chemotherapy in urinary bladder cancer with the main compounds available in the state of the art, after failure in BCG therapy, either by abandonment of treatment with BCG vaccine, or because of their high toxicity, remains highly experimental and so far did not achieve the same degree of success obtained with the use of BCG intravesical therapy.

The failures in BCG therapy, which is the best treatment available in the closest state of the art for urinary bladder cancer, either by its high toxicity that leads to premature abandonment, or by its relative ineffectiveness evidenced by the high rate of cancer recurrence, even for patients who completed the full treatment, clearly indicate the urgent need for improvements in the state of the art, through the provision of new treatments and drugs.

### Surgical Treatments (Cystectomy)

As previously mentioned, in the closest state of the art, the main therapy indicated for patients who do not respond to the best therapy available in the state of the art for bladder cancer, i.e., BCG intravesical therapy or chemotherapy following transurethral resection (TURBT), is partial or total removal of the urinary bladder due to the high risk of progression of cancer lesions to more invasive and/or metastatic forms.

This procedure is also indicated for patients who had invasive tumors (T2, T3, T4—TNM System) at the time of diagnosis. The total or partial removal of the urinary bladder, called cystectomy, is a high-risk surgical procedure and that usually involves a high degree of morbidity and mortality.

For instance, in a study with 1054 patients who underwent radical cystectomy due to bladder cancer, almost all had postsurgical complications of various degrees, including serious complications and death associated with this type of surgery. For reference and an example of the state of the art: Clark, Peter E. et al—Radical cystectomy in the elderly—Comparison of clinical outcomes between younger and older patients. Cancer-Volume 104, Issue 1, pages 36-43, 1 Jul. 2005.

### Alternative Treatments for Patients Who Cannot Undergo Surgery.

Due to the high probability of occurrence of morbidity or postsurgical complications, cystectomy is considered a high-risk procedure, and, thus, a large number of patients cannot undergo this type of surgery because of other pre-existing medical conditions and/or age-related conditions, such as heart problems, diabetes and others.

For patients who do not respond to standard therapy, i.e. surgical removal of lesions followed by intravesical chemotherapy (e.g., Adriamycin, Valrubicin) or immunotherapy (BCG, Interferon, IL-12), either due to medication intolerance (BCG), or failure to respond to therapy, and who have cancer recurrence, and additionally cannot undergo cystectomy, the existing alternatives are restricted to support measures, palliative measures using chemotherapy drugs (Valrubicin) and radiotherapy to try to prolong patients' lives of patients for as long as possible. For reference: Toni K Choueiri and Derek Raghavan. Chemotherapy for muscle-invasive bladder cancer treated with definitive radiotherapy: persisting uncertainties—Nature Clinical Practice Oncology (2008) 5, 444-454.

In the United States, a chemotherapy drug called Valrubicin (N-trifluoroacetyladriamycin-14-valerate) is topically used (intravesical therapy) for palliative treatment of patients with urinary bladder cancer who cannot undergo surgery (cystectomy).

Despite its low efficacy, as results are seen in only 20% of the patients treated, this drug (Valrubicin) is currently the only chemotherapeutic agent approved by the FDA for the palliative treatment of patients with bladder cancer resistant to topical treatment with BCG and who cannot undergo surgery for removal of the urinary bladder (cystectomy) because of comorbidities such as concomitant disease, advanced age and intolerance to anesthetic procedures. For reference: Dinney C P et, Greenberg R E, Steinberg G D. Intravesical valrubicin in patients with bladder carcinoma in situ and contraindication to or failure after bacillus Calmette-Guérin. Urol Oncol. 2013 November; 31(8):1635-42.

### Need for New Products and Therapies

Considering the evident shortcomings of the existing treatments in the closest state of the art for urinary bladder cancer, either surgical procedures (total or partial cystectomy, cryotherapy, laser), radiotherapy or drug treatments using chemotherapeutic compounds (ex: Thiotepa, Mitomycin C, Valrubicin) or immunotherapeutic compounds (BCG, exogenous interferons, IL-2, IL-12), there is urgent need in the state of the art for new modalities of treatment of epithelial cancer, including urinary bladder cancer, that could replace or complement the existing treatments, maximizing their efficiency.

It would be highly desirable that these new treatment modalities had no unwanted side effects commonly observed in the treatments available in the state of the art, as described in the present report.

The creative or inventive activity should therefore be aimed to the discovery of new therapeutic modalities, including new methods and products, used alone or associated, and which are comparatively more beneficial than the current treatments in terms of effectiveness and safety, with lower toxicity to the end user.

The present invention, in an innovative way, provides a new treatment of tumors of the superficial epithelium, including those located in the epithelium of the urinary bladder, through the use of an immunomodulator (proteic aggregate of ammonium and magnesium phospholinoleate-palmitoleate anhydride), which proved to be able to be used topically, alone, or in combination or association with the existing treatments, replacing them or reinforcing their effectiveness, and at the same time without the occurrence of unwanted side effects, which represents a significant advancement over existing treatments in the closest state of the art, as will be detailed in this report, with examples of practical use.

On the contrary, as will be shown in this report, the use of the referred substance, an immunomodulator, combined to the drugs in the closest state of the art, led to a noticeable decrease in the frequency of the unwanted side effects of these other treatments.

The present invention was derived from a compound already known in the state of the art and described in (PI-0305373-3, US 2006/0093628 A1, EP 1529784 A1, WO 2011082458A1), though used in a way that is entirely different from the previous forms of administration and use described in the state of the art, with outstanding and unprecedented practical results, as shall be detailed by means of descriptions and experiments in this report.

### The Invention

It is widely known in the state of the art that the best available strategy for treating tumors in the epithelial tissue lining surfaces outside and inside the body, especially tumors of the urinary bladder, is based on surgical removal of the tumor or affected tissue followed by adjuvant topical treatment with drugs, in order to avoid cancer recurrence or else to slow down its progression when complete elimination is not possible.

The use of compounds or drugs in the intravesical treatment of tumors located in the epithelial tissue lining surfaces outside and inside the body for topical application, such as urinary bladder tumors, is facilitated by the relatively unobstructed access to these anatomical regions, such as the skin and hollow organs as the urinary bladder.

This anatomical peculiarity allows access for topical application of compounds, by means of several techniques or special equipment, such as catheters.

In view of this peculiarity, any person skilled in the art can easily understand the advantages of topical treatment with drugs that act specifically on this type of tissue.

Finally, the topical use of drugs aims to ensure that the maximum safe therapeutic drug concentration reaches the most superficial epithelial surface where the tumors are located.

Therefore, considering the possibility of access to such regions, and the limited or no blood supply to these regions, the use of compounds with topical action is essential to ensure new effective treatments of malignant or premalignant lesions in the epithelial tissue lining outside or inside surfaces.

For example, the treatment of tumors in the bladder's internal lining benefits from the fact that the anatomy of the body provides access from outside for the introduction of drugs or solutions, with the use of catheters that penetrate the organ through the urethra and, then, accessing the inside lining of the urinary bladder.

This type of application, that is, the topical action of drugs in organs with cavities or interior spaces, can be and is used for the treatment of cancers in the lining epithelium of other organs, besides the urinary bladder, such as the uterus, stomach or esophagus.

Given these peculiarities and the problems cited, and for the purposes of the invention, that is, providing a novel and effective therapeutic agent capable of topical action in the epithelial tissue lining outside and inside surfaces of body organs, a compound with the following properties and capabilities was required:

A) can be used for therapeutic effects against malignant or premalignant lesions located both in the epithelium lining outside and inside surfaces;
B) capable of producing therapeutic effect alone or combined to other drug and non-drug treatments;
C) no significant toxicity;
D) capable of eliminating or minimizing toxic effects of other treatments.

The invention, in an innovative and remarkable way, as shall be detailed in this report, with examples of its practical use, allows meeting all these requirements, that is, the above mentioned items A, B, C and D.

The invention was developed based on new and surprising scientific data from the use of invention in human beings, and also experiments with animals involving comparative assessment of topical treatments for epithelial cancer, including urinary bladder cancer.

These experiments involved the comparison in animal models of several compounds already known in the state of the art, including BCG vaccine, chemotherapeutic drugs such as Mitomycin C and Valrubicin, and also the proteic aggregate of ammonium and magnesium phospholinoleate-palmitoleate anhydride described in the state of the art in PI-0305373-3, US 2006/0093628 A1, EP 1529784 A1, WO 2009/097670A1, WO 2011082458 A1.

Analysis of data of these new experiments and scientific studies revealed effects and biological and therapeutic properties entirely unprecedented in the state of the art for the immunomodulator proteic aggregate of ammonium and magnesium phospholinoleate-palmitoleate anhydride. These new unprecedented properties in the state of the art were incorporated in the present invention.

In animal models, when applied in epithelial tissues affected by cancer, the immunomodulator (proteic aggregate of ammonium and magnesium phospholinoleate-palmitoleate anhydride) in an outstanding and unprecedented way showed, in clinical practice, its effective topical action on the epithelial region affected by the cancer.

This unprecedented property of the referred substance (proteic aggregate of ammonium and magnesium phospholinoleate-palmitoleate anhydride), that is, its ability (until now unknown in the state of the art) of being applied directly where its action is desired, has allowed its use in the present invention as topical medication for the treatment of malignant or premalignant lesions that affect the epithelial tissue lining outside or inside surfaces.

Additionally, the ability of the immunodulator (proteic aggregate of ammonium and magnesium phospholinoleate-palmitoleate anhydride) when used alone in the present invention for the topical treatment of malignant lesions, represents a major advance compared to the closest state of the art, since it proved to be much more effective than the main drugs treatments used in the state of the art, when compared to the latter in practical uses (Tables 4, 5, 6-A, 6-B, 7, 8-A, 8-B).

In a remarkable and unprecedented way, the immunomodulator (proteic aggregate of ammonium and magnesium phospholinoleate palmitoleate anhydride) not only maximized the effect of other drugs already used in the state of the art (Tables 4, 5, 6-A, 6-B) when used in combination or association, and also when topically applied, but also significantly decreased the toxicity of treatment for the end user, in the association, with these remarkable and unprecedented practical advantages of its use being added for the purposes of the invention.

When a combination of drugs or treatments is used in clinical practice with a biological or therapeutic effect that is wider than the effects of its isolated components, this remarkable event is called synergy or synergistic effect of the referred combination or association.

In the case of association of the immunomodulator (proteic aggregate of ammonium and magnesium phospholinoleate-palmitoleate anhydride) with other drugs, including BCG vaccine, Mitomycin C and Valrubicin, in the topical treatment of urinary bladder cancer, the synergy or synergistic effect provided by the invention, consists in the maximization of the therapeutic effect, combined with a remarkable decrease in toxicity for the association. (Tables 4, 5, 6-A).

This unprecedented ability of the immunomodulator (proteic aggregate of ammonium and magnesium phospholinoleate-palmitoleate anhydride) of acting topically on the epithelial tissue lining outside and inside surfaces, and without toxicity for the region treated, allows it to be used as topical treatment of malignant and premalignant lesions, alone or in association or combination with other therapies and procedures, with remarkable advantages compared to the treatments that were known until now in the state of the art.

### Absolute Novelty of the Invention—Topical Use of the Immunomodulator Proteic Aggregate of Ammonium and Magnesium Phospholinoleate-Palmitoleate Anhydride for the Treatment of Cancer in the Epithelial Tissue—State of the Art

Until now, it was reported in the state of the art (PI-0305373-3 US 2006/0093628 A1, EP 1529784 A1, WO 2011082458A1) that the immunomodulator (proteic aggregate of ammonium and magnesium phospholinoleate-palmitoleate anhydride) had antitumor activity only when it acted systemically, that is, by means of stimulation and/or modulation of the immune system, and this effect would only occur when the compound was introduced into the body by parenteral route, using subcutaneous, intramuscular or intraperitoneal injections.

Or, in other words, the immunomodulator (proteic aggregate of ammonium and magnesium phospholinoleate-palmitoleate anhydride) was always administered as an injectable drug for the treatment of cancer and other diseases, and once injected into the body would act systemically by stimulating the immune system.

In fact, in PI-0305373-3 US 2006/0093628 A1, EP 1529784 A1 and in other references in the state of the art (WO 2009/097670 A1, WO 2011082458A1), it is reported that the immunomodulator (proteic aggregate of ammonium and magnesium phospholinoleate-palmitoleate anhydride) has systemic therapeutic action, and, therefore, should be administered by injection (intramuscular, intraperitoneal and subcutaneous) in the body.

However, the best therapeutic strategy available in the closest state of the art for the treatment of urinary bladder cancer, as well as other tumors affecting the epithelial tissue lining outside or inside surfaces, also requires that topical treatment is adopted after surgical removal of the tumor, in order to prevent the recurrence of cancer.

This particular form of treatment that represents the closest state of the art for the treatment of tumors affecting the epithelial tissue of outside and inside surfaces of the body uses drugs with topical action based on the fact that compounds that act systemically and/or depend on internal transport routes, such as the circulatory or lymphatic system, are ineffective when used in the treatment of malignant or premalignant lesions in the epithelial tissue lining inside or outside surfaces of the body.

The explanation for this phenomenon that obstructs or neutralizes the benefits of drugs with systemic action is that the superficial regions of the internal and external epithelium have limited blood supply, which hinders or even prevents the access of an adequate concentration of drugs to the site of the lesion (cancer), when these medications depend on internal transportation routes, e.g. injectable drugs, as will be detailed in the present report.

As reported in the state of the art, the immunomodulator (proteic aggregate of ammonium and magnesium phospholinoleate-palmitoleate anhydride) was always ineffective when used in cancer cells in vitro, or in other words, this ability to be applied directly to the region where its action is desired was entirely unknown until now in the state of the art.

Therefore, we cite US 2006/0093628 A1: “Cytotoxic properties. Cytotoxicity of the compound of the present invention was studied in the range of 10⁻⁴ to 10⁻⁸ M in 53 tumor cell cultures representing 8 distinct tumor types (lung, colon, central nervous system, melanoma, and ovary, kidney, prostate and mammary) and leukemia. The compound of the present invention did not display cytotoxicity for these tumor cells.”

Or else, the immunomodulator (proteic aggregate of ammonium and magnesium phospholinoleate-palmitoleate anhydride) in the state of the art had no topical action against cancer cells, once when directly applied to regions with several tumor strains (US 2006/0093628 A1, EP 1529784 A1) showed no toxicity, that is, was unable to damage or eliminate cancer cells when topically applied.

Therefore, the immunomodulator (proteic aggregate of ammonium and magnesium phospholinoleate-palmitoleate anhydride) has always been used as a systemic drug injected into the body, generating an effective immune response against cancer and also intracellular parasites. (US 2006/0093628 A1, EP 1529784 A1, WO2011082458 A1, WO 2009/097670 A1).

Nevertheless, when the immunomodulator (proteic aggregate of ammonium and magnesium phospholinoleate-palmitoleate anhydride) was used in recent experiments in animal models for the study of urinary bladder cancer, for the purpose of comparison with drugs with topical action, including chemotherapy and immunotherapy drugs (BCG vaccine). In these experiments, the immunomodulator was used in the injectable form and topically applied at the same concentrations. (Tables 4, 5, 6-A, 6B)

In a remarkable and unprecedented way, contrary to all previous information in the state of the art, the substance was found to have potent antitumor action when introduced by a catheter in the epithelial tissue lining the urinary bladder of experimental animals (Table 4, Table 5, Table 6-A, Table 6-B).

In one of the above cited experiments, the immunomodulator (proteic aggregate of ammonium and magnesium phospholinoleate-palmitoleate anhydride) was also experimentally used, in its injectable form, in the animal model for the study of bladder cancer, and showed no comparable results at the concentrations used (Table 4—Group E) when compared to the same compound, though topically applied. (Table 4—Group-D).

Now, in a remarkable way, the immunomodulator (proteic aggregate of ammonium and magnesium phospholinoleate-palmitoleate anhydride) that was reported in the state of the art as being effective against infections and cancer when systemically administered by injection, has shown topical action against cancer in the inner lining (epithelium) of the bladder (Tables 4,5,6-A, 6-B).

This unprecedented action, that is the topical action of the immunomodulator (proteic aggregate of ammonium and magnesium phospholinoleate-palmitoleate anhydride) against cancer affecting the epithelial tissue, when topically used, that is, without the need to be administered by injection, was incorporated to the present invention. This unprecedented ability will be detailed in the present report by means of examples of its practical use in animals and also in trials with humans.

The immunomodulator (proteic aggregate of ammonium and magnesium phospholinoleate-palmitoleate anhydride), in the way it was used in the present invention, showed therapeutic properties when directly applied to the affected epithelial tissue, e.g. after being introduced in the bladder of experimental animals (Tables 4,5,6-A, 6-B), through a catheter, added to saline suspension or else in cream formulation to be topically applied in humans (Table 7, 8-B) and other formulations (Table 6-B), as described, also with examples of its practical use in the present report.

These results are totally unprecedented and remarkable, once all data from the scientific literature and other information available in the state of the art so far provided indications that the product (proteic aggregate of ammonium and magnesium phospholinoleate-palmitoleate anhydride) was only effective against cancer and other diseases when systemically administered, that is, indirectly acting by triggering a immune response of the body after being injected in animals and humans.

And also, when used for the treatment of urinary bladder cancer, the immunomodulator (proteic aggregate of ammonium and magnesium phospholinoleate-palmitoleate anhydride) was found to be more effective than the best topical treatment available in the state of the art for urinary bladder cancer, which is BCG vaccine, as will be demonstrated in the present report by means of examples of its practical use.

Finally, and also in a remarkable way, the immunomodulator (proteic aggregate of ammonium and magnesium phospholinoleate-palmitoleate anhydride) did not show adverse side effects in the inner mucosa (epithelium) of the organ, compared to the most advanced intravesical medication in the state of the art for the treatment of bladder cancer at high risk of progression (Ta, T1, C is), that is, BCG vaccine (Tables 4, 5, Table 6-A).

While all animals with urinary bladder cancer treated with BDG vaccine showed urinary bleeding of post-treatment hematuria, the animals treated only with the immunomodulator (proteic aggregate of ammonium and magnesium phospholinoleate-palmitoleate anhydride) showed no sign of urinary bleeding. Also, microscopic examinations of the tissues of the animals revealed signs characteristic of cancer reversion. (Table 4, Table 5, Table 6-A, Table 6-B).

Reversion of the tumor process, that is, the curative effect, occurred when the immunomodulator (proteic aggregate of ammonium and magnesium phospholinoleate-palmitoleate anhydride) was used alone and also associated to other compounds in the treatment of urinary bladder cancer, maximizing the therapeutic effectiveness of the association, and even eliminating hematuria, which is demonstrated with an example of the practical use of the invention in animal model (Table 5 and Table 6-A).

In this particular aspect, or else, when associated to other drugs such as BCG, Mitomycin C and Valrubicin, the use of the immunomodulator (proteic aggregate of ammonium and magnesium phospholinoleate-palmitoleate anhydride) not only proved to be able to maximize the effectiveness against cancer lesions in the inner epithelium (urinary bladder), but also, in a more remarkable way, created a synergistic effect for the association, since it remarkably decreased the toxicity associated to the treatment, which is demonstrated by the elimination of hematuria in the experimental animals (Table 5 and Table 6-A).

The reversion of premalignant and malignant lesions in the epithelium lining internal and external surfaces (skin) in humans, is also sufficiently demonstrated in the present report, so that any expert with knowledge of the state of the art understands that the invention can be used without any obstacle in the treatment of malignant or premalignant lesions of this anatomical region, with the benefits of topical application, which is also one of the purposes of the present invention (Tables 7 and Tables 8-A and 8-B).

### Purposes of the Invention

Therefore, one of the purposes of the present invention is to provide a compound for the topical adjuvant treatment of malignant and premalignant lesions in the epithelial tissue lining surfaces inside and outside the body.

Another purpose of the present invention is to provide a specific compound for topical treatment of tumors in the urinary bladder, which was found to be more effective than the main drugs in use in the state of the art for this type of disease.

Another purpose of the invention is also to provide a compound to be used in association or combination with other types of treatments, including drug or non-drug treatments, intended for treatment of malignant and premalignant lesions in the epithelial tissue lining inside and outside surfaces.

The present invention aims to provide a new and powerful treatment able to maximize therapeutic effectiveness when used in combination or association other drugs in the state of the art intended for topical treatment of malignant and premalignant lesions in the epithelial tissue lining inside and outside surfaces of the body.

The present invention also aims to provide a new drug treatment without significant toxicity for users compared to drugs used in the state of the art intended for topical treatment of malignant lesions in the epithelial tissue lining inside and outside surfaces of the body.

Additionally, the present invention also aims to provide a new and powerful therapy able to create a synergistic therapeutic effect when combined to other drugs in the state of the art, intended for topical treatment of malignant lesions in the epithelial tissue lining inside and outside surfaces, due to its ability to maximize the therapeutic effectiveness of the combination or association, and simultaneously reduce the toxicity of the association for end users.

The present invention also aims to provide a new and powerful therapy able to replace the existing drug treatments, including BCG vaccine, when used in the topical treatment of malignant lesions in the epithelial tissue lining inside surfaces, in the event that a satisfactory result is not obtained with BCG vaccine, or else, such treatment needs to be replaced by other treatments due to unacceptable toxicity levels, which are intolerable to users.

The present invention also aims to provide a new palliative treatment against malignant lesions in the epithelial tissue lining inside and outside surfaces to be used when, in the event that the medication fails, more aggressive treatments or that related to at higher risk of morbidity and mortality, such as surgical removal of the urinary bladder (cystectomy) or other forms of treatments that eliminate lesions, cannot be performed due to unacceptable risk to the patients.

The present invention also aims to provide topical formulations, using pharmaceutically acceptable carriers for use in treatment of the malignant or premalignant lesions in subjects in need thereof.

Finally, in order to sufficiently demonstrate the properties and benefits of the invention compared to the main products existing in the state of the art, several examples of its practical use are reported in this specification (Table 4, Table 5, Table 6-A, Table 6-B, Table 7, Table 8-A, Table 8B).

These experiments of the practical use of the invention and its results are detailed in the present report to demonstrate the inventive activity, the absolute novelty and the uses and comparative benefits of the invention. These experiments are presented for illustrative purposes only, and by no means intend to limit the scope and the field of application of this invention.

Without the need for additional information, it can be affirmed that any person skilled in the art, based only on the information and examples of its practical use that will be provided in this report, should be able to understand and use the present invention to its full extent.

Although the invention is intended for human use, it can be also used in the treatment of malignant or premalignant lesions in animals, which also have epithelial tissue that lines inside and outside surfaces of their bodies and can also be affected by tumors in these anatomical regions.

Practical Example of Use of the Invention in the Treatment of Bladder Cancer—Comparison with BCG Vaccine—Experiment No. 1—Table 4
Objective: Comparative assessment of the therapeutic response in animals subjected to experimental induction of bladder cancer with n-methyl-n-nitrosourea (MNU)
Fifty animals (n=50) were used in the experiment.

Of these, 40 animals were given 1.5 mg/kg of n-methyl-n-nitrosourea (MNU) dissolved in 0.30 ml of sodium citrate, intravesically, with the use of a catheter, for 7 weeks. One group (control—Group-A) of 10 animals was only given saline solution (NaCl 0.9,%) also intravesically and for an equal period. The compound n-methyl-n-nitrosourea (MNU) was applied in the animals to experimentally induce immunosuppression and the subsequent production of urinary bladder carcinoma. After treatment with MNU, the animals were divided into 05 groups of 10 animals each.

After the use of MNU for 7 weeks, 4 batches of 10 animals each were subsequently subjected to intravesical instillation of 0.30 ml of 0.9% saline solution (GROUP-B—MNU), 10⁶ CFU (40 mg) of intravesical BCG (Group-C) and 10 mg/kg of the immunomodulator (proteic aggregate of ammonium and magnesium phospholinoleate-palmitoleate anhydride—Group-D), suspended in saline, once a week, for all the groups, using a catheter, during the entire treatment period of 14 weeks.

For the purposes of comparison, 10 mg/kg of the immunomodulator (proteic aggregate of ammonium and magnesium phospholinoleate-palmitoleate anhydride) was also used, however administered as a subcutaneous injection (Group-E), once a week during the entire treatment period (14 weeks).

After this procedure, 10 animals of each group were sacrificed and their urinary bladders removed for collection of tissue fragments, which were embedded in paraffin and prepared for histological section, by the usual techniques.

For the histological examinations, sections (2 μm) of the tissue of the urinary bladder were cut using microtome Zeiss, mounted on microscope slides, and stained with hematoxylin-eosin (HE), for pathology assessment, quantification of cellular changes and comparison between control and treatment groups.

Finally, blood from treated and untreated animals was collected at the end of the experiment, processed, and the plasma separated from blood cells was fully digested with the use of concentrated acids (H₂SO₄ and HNO₃) and subjected to techniques for assessing the amount of Mg++, which is the main inorganic component of the molecule (proteic aggregate of ammonium and magnesium phospholinoleate-palmitoleate anhydride) by atomic absorption for verification and quantification of a possible systemic absorption of the compound of the invention from the lining epithelial tissue.

This particular assessment, that is, assessment of the amount of Mg++ in the plasma of experimental animals was only performed in the experiments reported in Table 4 (Table 4—Groups A and D).

### Criteria Adopted for Assessment of Cellular Changes in Urinary Bladder Cancer and Toxic Effects of Treatments—Table 4, Table 5, TABLE 6-A and TABLE 6-B.

The therapeutic effect in urinary bladder cancer in animals was assessed by histological evaluation of the tissues. The criteria for assessing normality of the tissues examined for all the experiments, that is, the experiments reported in Table 4, Table 5, Table 6-A, Table 6-B are: normal tissue, flat hyperplasia, papillary hyperplasia.

Flat hyperplasia and papillary hyperplasia are reactive changes of an inflammatory nature, though benign, and their occurrence in the experiments for assessment of new drugs in animals affected with cancer, indicates a reversal of the cancer process to non-cancerous stages.

The criteria adopted in all the experiments involving urinary bladder cancer in the present report, that is, the experiments contained in tables 4,5 and 6-A and 6-B for the presence of malignant cellular changes (cancer) and their degrees of severity are: low grade intraepithelial neoplasia, high grade intraepithelial neoplasia (carcinoma in situ-Tis), papillary carcinoma (Ta) and squamous metaplasia.

Metaplasia is defined as the replacement of a normal functional tissue with another tissue with different structure, with loss of function, and, thus, is considered a form of premalignant lesion.

The same criterion was adopted for all experiments involving urinary bladder cancer contained in Tables 4,5,6-A and 6-B, for assessment of adverse side effects (toxic effect) of the treatments, that is, the presence or absence of hematuria (bleeding) in the animals.

In the experiment reported in Table 4, below, this assessment was performed in all animals and compounds used: (proteic aggregate of ammonium and magnesium phospholinoleate-palmitoleate anhydride) topical/intravesical (Group D—Table 4), (proteic aggregate of ammonium and magnesium phospholinoleate palmitoleate anhydride injectable (Group E—Table 4) and BCG—topical formulation (Group C—Table 4) and the results are shown in Table 4.

TABLE 4
Histological evaluation

| | | Group B | | Group D | Group E |
|---|---|---|---|---|---|
| | | (MNU) + | | (Intravesical | (Injectable |
| | Group A (non | saline | Group C (BCG) | polymeric | polymeric |
| | treated control) | solution | intravesical | anhydride) | anhydride) |
| | n = 10 | n = 10 | n = 10 | n = 10 | N = 10 |
| Flat | — | — | — | 06 (60%) | — |
| hyperplasia | | | | | |
| Papillary | — | 01 = 10% | 0 = 40% | 02 = 20% | 02 (20%) |
| hyperplasia | | | | | |
| Low grade | — | | 03 = 0% | — | 04 (40%) |
| Intraepithelial | | | | | |
| neoplasia | | | | | |
| High-degree | — | 03 = 30% | 02 = 20% | — | 01 (10%) |
| intra-epithelial | | | | | |
| carcinoma | | | | | |
| (carcinoma in | | | | | |
| situ-Tis) | | | | | |
| Papillary | — | 06 = 60% | 01 = 10% | — | 01 (10%) |
| carcinoma | | | | | |
| (Ta) | | | | | |
| Squamous | — | — | 01 = 10% | — | 02 (20%) |
| metaplasia | | | | | |
| Normal | 100% | — | — | 2 = 20% | — |
| Toxicology: | Negative | Positive | Positive | Negative | Positive |
| (Hematuria) | (100%) | (100%) | (100%) | (0%) | (100%) |
| Mg++/Plasma | 34.6 ± 5.1 | — | — | 32.6 ± 4.2 | — |
| (ug/mL) | | | | | |

### Discussion of the Results—Table 4

The therapeutic effect of the topical use (intravesical) of the compound of the present invention (proteic aggregate of ammonium and magnesium phospholinoleate-palmitoleate anhydride), in the practical example provided, that is, treatment of cancer induced in rats by MNU was much higher compared to the best therapy available in the state of the art (BCG) for treating malignant lesions of the epithelium of the urinary bladder.

This therapeutic effect was assessed for all the compounds used, by means of histopathological analysis of the epithelial tissue of the urinary bladder. (Table 4) The experiment showed a positive result for practically 100% of the animals treated with the compound of the invention, that is, the proteic aggregate of ammonium and magnesium phospholinoleate-palmitoleate anhydride when topically used (Table 4—Group D), with the epithelial tissue of 20% being completely normalized and 60% of the animals showing only flat hyperplasia and 20% with papillary hyperplasia.

Because hyperplasia is a non-cancerous condition, based on the experiment reported in Table 4, it can be concluded that the invention shows a therapeutic effectiveness of 100% (with 60% for hyperplasia and 20% for normality—Table 4—Group D) compared to the MNU group (Group B—Table 5) in which 90% of the animals had cancer (30% classified as in situ-T is carcinoma and 60% as papillary carcinoma-Ta.

However, the use of the immunomodulator (proteic aggregate of ammonium and magnesium phospholinoleate-palmitoleate anhydride—Table 4—Group E), in the injectable form, as subcutaneous injections, at the same concentrations (10 mg/kg) did not show comparable therapeutic effects when used in the topical form, which can be due to the low absorption capacity of the inner tissue of the urinary bladder regarding drugs systemically administered that can only be introduced in the body by parenteral route, as previously described. Only 20% of the treated animals by subcutaneous injection showing papillary hyperplasia and the remaining treated animals showing low grade intraepithelial neoplasia (40%), (10%) carcinoma in situ (Tis), (10%) Papillary carcinoma (Ta) and (20%) squamous metaplasia (Table 4—Group E).

The topical use of BGC vaccine showed a positive outcome for 40% of the animals, (Table 4—Group C) that showed papillary hyperplasia, while 20% had high grade neoplasia (carcinoma in situ (Tis)-TNM classification) and 10% of the animals had squamous metaplasia (Table 4—Group C).

Although hyperplasia is a non-cancerous condition, metaplasia, as previously cited, is defined as replacement of a normal functional tissue by another tissue with different structure, with loss of function, and, thus, is also considered a form of premalignant lesion.

Regarding the amount of magnesium, the main inorganic component of the proteic aggregate of ammonium and magnesium phospholinoleate-palmitoleate anhydride measured in the plasma of the experimental animals, the quantitative assessment of Mg++ ions showed equal results for the untreated control group (Group A—Table 4) and the treated group (Group D—Table 4), that is, 34.6±5.1 ug/mL. Such data indicates that there was no systemic absorption of the compound of the invention (proteic aggregate of ammonium and magnesium phospholinoleate-palmitoleate anhydride), when topically used (Table 4—Group D).

The non-penetration of magnesium, the main inorganic component of the proteic aggregate of ammonium and magnesium phospholinoleate—palmitoleate anhydride beyond the epithelial layer (Table 4—group D) is possibly due to the relatively large size of the molecule (proteic aggregate of ammonium and magnesium phospholinoleate palmitoleate anhydride), and/or because of its high molecular weight, which is 320.000 Dalton (320 kDa).

This above cited finding demonstrates that the biological and antitumor therapeutic effect of the immunomodulator (proteic aggregate of ammonium and magnesium phospholinoleate-palmitoleate anhydride) in the case is due to a local antitumor effect in the lining epithelium of urinary bladder. (Table 4—Group D)

Finally, the animals given the carcinogenic compound MNU (Table 4—Group B) and which were not treated with any drug following the application of MNU, almost all of the them (90%) had cancerous lesions, with 60% developing papillary carcinoma (Ta—TNM classification) and 30%, high-grade carcinoma in situ (Tis-TNM classification), indicating that the referred compound (MNU) is highly effective in the experimental induction of cancerous lesions. Furthermore, in all the animals treated with MNU blood was detected in the urine (hematuria)

### Toxicology:

Animals treated with the invention: None of the animals in the experiment that was given the compound of the present invention, that is, the immunomodulator (proteic aggregate of ammonium and magnesium phospholinoleate-palmitoleate anhydride) in continuous topical application (Group D—Table 4) showed signs of urine blood (hematuria).

Animals treated with BCG: In all the animals treated with BCG blood was detected in the urine (hematuria). The degree of hematuria in these animals ranged from moderate (80%) to severe (20%) in response to continuous intravesical application of BCG from the second week. (Group C—Table 4)

Animals treated with injectable immunomodulator (Group E—Table 4): All the animals treated with the immunomodulator (proteic aggregate of ammonium and magnesium phospholinoleate-palmitoleate anhydride) in the injectable form were found to have blood in the urine (hematuria), indicating the low effectiveness of the treatment by this route of administration.

In all the experiments involving the administration of the invention in the treatment of urinary bladder cancer, hematuria was used as parameter and the most evident sign for assessment of the local toxicity of the compounds used, for it is related to chronic irritation or inflammation of the affected tissue (epithelium lining the urinary bladder), by topically applied drugs (e.g. BCG vaccine) and also with epithelial tissue's inflammation related with the cancerous process.

Therefore, the use of the present invention in the practice is also more effective than immunotherapy with BCG regarding toxicity too, since the result of the practical experiment undoubtedly showed that the use of the present invention did not cause any sign of toxicity in the epithelium (Group D—Table 4) compared to BCG (Group C—Table 4) for equal periods and using the same route of administration, i.e. intravesical.

Consequently, and in a remarkable way, the compound used for the purposes of the present invention (proteic aggregate of ammonium and magnesium phospholinoleate-palmitoleate anhydride) is not only capable of topical action, which is unprecedented in the state of the art, but is also comparatively more effective in the treatment of bladder cancer than the best therapy available so far in the state of the art, i.e. BCG vaccine.

The results of the use of the invention shown in this example of practical use, when compared to BCG, regarding therapeutic effectiveness and assessment of unwanted side effects, measured for the presence or absence of hematuria, demonstrate that the invention represents a significant advance compared to the therapies in the closest state of the art in the treatment of urinary bladder cancer.

### Second Practical Experiment—Use of the Present Invention Compared to Chemothyerapy Drugs in the Intravesical Treatment of Bladder Cancer—(TABLE-5)

Fifth animals (n=50) were used in the experiment.

The therapeutic effect was assessed through histological assessment of tissues, and the toxicological effect of the compounds, through assessment of the presence or absence of urinary bleeding also called hematuria. The results are shown in Table 5.

The following compounds were used in this experiment: proteic aggregate of ammonium and magnesium phospholinoleate-palmitoleate anhydride (PI-0305373-3, US 2006/0093628 A1, EP 1529784 A1), Mitomycin C, Valrubicin, topically administered for comparative assessment in the treatment of MNU-induced cancerous lesions.

All the animals in the experiment were subjected to intravesical applications of 1.5 mg/kg of n-methyl-n-nitrosourea (MNU) dissolved in 0.30 mL of sodium citrate, using a catheter, for 7 weeks. One group (control) of 10 animals was given only saline solution (NaCl 0.9%), also intravesically and for an equal period.

The purpose of using n-methyl-n-nitrosourea (MNU) was to experimentally induce immunosuppression and subsequently producing urinary bladder carcinoma. After treatment with MNU, the animals were divided into 05 groups of 10 animals each.

After the use of MNU for 7 weeks, 5 batches of 10 animals each were intravesically inoculated with 0.30 ml of 0.9% saline solution (control group), 10 mg/kg of the chemotherapeutic agent Mitomycin C suspended in sterile water for injection, 10 mg/kg of the chemotherapeutic agent Valrubicin, suspended in diluents supplied by the manufacturer and 10 mg/kg of the immunomodulator (proteic aggregate of ammonium and magnesium phospholinoleate-palmitoleate anhydride), suspended in saline solution, using intravesical catheter for the topical application of all the substances during 14 weeks.

After this procedure, all the 10 animals of each group were sacrificed and their urinary bladders were removed for collection of tissue fragments, which were embedded in paraffin and prepared for histological section, by the usual techniques.

For the histological examinations, sections (2 μm) of the tissue of the urinary bladder were cut using microtome Zeiss, mounted on microscope slides, and stained with hematoxylin-eosin (HE), for subsequent pathology assessment, quantification of cellular changes and comparison between control and treatment groups. The criteria for assessment of these lesions, the effects of treatment and the assessment of toxic effects (hematuria) have been previously described above in the specification.

The comparative therapeutic and toxicity findings regarding the use of the invention, Mitomycin C and Valrubicin are shown in Table-5, below.

TABLE 5
Histological assessment

| | Group A | | | Group D | Group E |
|---|---|---|---|---|---|
| | (untreated | Group B | Group C | Polymeric | (Valrubicin) |
| | control) | (MNU) | (Mitomycin D) | anhydride) | N = 10 |
| | n = 10 | n = 10 + | n = 10-topical | n = 10 topical | topical |
| | NaCl 0.9% | NaCl 0.9% | application | application | application |
| Flat | — | — | — | 05 (50%) | — |
| hyperplasia | | | | | |
| Papillary | — | 01 = 10% | 4 = 40% | 02 = 20% | — |
| hyperplasia | | | | | |
| Low grade | — | | 03 = 30% | — | 5 (50%) |
| intraepithelial | | | | | |
| neoplasia | | | | | |
| High grade | — | 03 = 30% | 02 = 20% | — | 2 (20%) |
| intraepithelial | | | | | |
| neoplasia | | | | | |
| (carcinoma in | | | | | |
| situ-Tis) | | | | | |
| Papillary | — | 06 = 60% | — | — | 1 (10%) |
| Carcinoma | | | | | |
| (Ta) | | | | | |
| Squamous | — | — | — | — | 1 (10%) |
| metaplasia | | | | | |
| Normal | 100% | — | 01 = 10% | 3 = 30% | 1 (10%) |
| Toxicology: | Negative | — | Positive | Negative | Positive |
| (Hematuria) | (0%) | | (100%) | (0%) | (100%) |

### Discussion of the Results—Table 5

Assessment of the data contained in Table-5 clearly shows that the use of the immunomodulator (proteic aggregate of ammonium and magnesium phospholinoleate-palmitoleate anhydride) alone, topically administered, i.e., directly applied (intravesically) to the epithelial tissue lining the urinary bladder, affected by cancer, had a more significant therapeutic effect, either regarding tumor response or lower toxicity (Group D, Table 5), compared to the group treated with the compound Mitomycin C (Group C—Table 5) and also with Valrubicin (Group E—Table 5).

While in the group treated with Mitomycin C only 10% of the animals were within normal parameters, 40% with papillary hyperplasia and 50% still had cancer (Group C—Table-5), in the group treated with intravesical topical application of the compound of the invention (proteic aggregate of ammonium and magnesium phospholinoleate palmitoleate anhydride) 30% of the animals were within normal parameters and 70% had only hyperplasia. (Group D—Table 5). In the group treated with Valrubicin (Group E—Table 5) only 10% had tumor regression (normal animals) after treatment.

Since hyperplasia is a non-cancerous condition, based on the experiment reported in Table 5, it can be concluded that the invention had a therapeutic effectiveness of practically 100% (with 70% for hyperplasia and 30% for normality) compared to the control group (Group B—Table 5) in which 90% of the animals had cancer (30% classified as in situ-T is carcinoma and 60% as papillary carcinoma-Ta).

Regarding Mitomycin C (Group C—Table 5), it showed an efficacy of 40% (Papillary hyperplasia), 50% of the animals had cancer (30% with low-grade carcinoma and 20% with carcinoma in situ-Tis), with only 10% of the animals within normal parameters (Group C—Table 5). Finally the group treated with Valrubicin (Group E—Table 5) showed an effectiveness of only 10% (normal animals).

Regarding toxicology, it is immediately noted that the topical use of the compound of the invention did not result in any local toxicity measured by the presence of hematuria (Group D-Table 5), since none of the animals treated had urinary bleeding (Group D—Table 5), while the animals that used Mitomycin C were 100% positive for the presence of hematuria (Group C—Table 5) as well the animals treated with Valrubicin, i.e., 100% of the treated animals (Group E—Table 5).

Third Practical Experiment—Assessment of the Combined Use of the Invention with Chemotherapy and Immunotherapy Drugs—Table 6-A

Ninety animals (n=90)—Fischer 344 rats were used in the experiment.

The compounds proteic aggregate of ammonium and magnesium phospholinoleate-palmitoleate anhydride (10 mg/kg), BCG (10⁶ CFU (40 mg)) Mitomycin C (10 mg/kg), Valrubicin (10 mg/kg) were used in this experiment, all intravesically administered using a urinary catheter, for comparative assessment in the treatment of MNU-induced cancerous lesions (Table 6-A).

All the animals (Fisher 344 rats), except for the untreated control group (n=10), received intravesical inoculations (1.5 mg/kg) of n-methyl-n-nitrosourea (MNU) dissolved in 0.30 ml of sodium citrate, using a catheter, for 7 weeks. One group (untreated control group) of 10 animals received only saline solution (NaCl 0.9%), also intravesically, and for an equal period. After treatment with MNU, the animals were divided into 07 groups of 10 animals each.

After receiving MNU for 7 weeks, the 9 batches of 10 animals each were subsequently subjected to intravesical inoculation of the compounds of interest during 8 weeks in total.

The groups were divided, as follows: Group A (untreated control) received only saline solution (NaCl at 0.9%) for 8 weeks; group B received only MNU and 0.9% saline; group C received 10 mg/kg of the compound of the invention (proteic aggregate of ammonium and magnesium phospholinoleate-palmitoleate anhydride (10 mg/kg), for 8 weeks, intravesically; group D received BCG vaccine (10⁶ CFU (40 mg)) for 4 weeks, followed by the application of the compound of the present invention (proteic aggregate of ammonium and magnesium phospholinoleate-palmitoleate anhydride—10 mg/kg) for another 4 weeks.

Group E received Mitomycin C (10 mg/kg) for 4 weeks, followed by the application of the compound of the present invention (proteic aggregate of ammonium and magnesium phospholinoleate-palmitoleate anhydride 10 mg/kg) for another 4 weeks; group F received BCG vaccine (10⁶ CFU (40 mg)) for 8 weeks and group G received Mitomycin C (10 mg/kg) for 8 weeks. Group H received Valrubicin (10 mg/kg) for 8 weeks and Group I received Valrubicin (10 mg/kg) for 4 weeks, followed by application of the compound of the present invention (proteic aggregate of ammonium and magnesium phospholinoleate-palmitoleate anhydride (10 mg/kg) for another 4 weeks.

All the compounds were topically administered, by intravesical instillation, in the animals, using the appropriate diluents already described in the present report. For better comprehension of the experiment, see the table below, called Experimental Groups.

TABLE

| Experimental Groups | Compounds and periods of use |
|---|---|
| Group A (n = 10) | Saline 0.9% - Untreated control group |
| Group B (n = 10) | MNU + saline 0.9% - 8 weeks |
| Group C (n = 10) | proteic aggregate of ammonium and |
| | magnesium phospholinoleate-palmitoleate |
| | anhydride for 8 weeks (10 mg/kg) |
| Group D (n = 10) | BCG vaccine (10⁶ CFU (40 mg)) for 4 |
| | weeks plus |
| | proteic aggregate of ammonium and |
| | magnesium phospholinoleate-palmitoleate |
| | anhydride for 4 weeks (10 mg/kg) |
| Group E (n = 10) | Mitomycin C (5 mg/kg) for 4 weeks plus |
| | proteic aggregate of ammonium and |
| | magnesium phospholinoleate-palmitoleate |
| | anhydride for 4 weeks (10 mg/kg) |
| Group F (n = 10) | BCG vaccine (10⁶ CFU (40 mg)) for 8 |
| | weeks |
| Group o G (n = 10) | Mitomycin C (10 mg/kg) for 8 weeks |
| Group H (n = 10) | Valrubicin (10 mg/kg) for 8 weeks |
| Group I (n = 10) | Valrubicin (10 mg/kg)-4 weeks plus |
| | proteic aggregate of ammonium and |
| | magnesium phospholinoleate-palmitoleate |
| | anhydride (10 mg/kg) for 4 weeks |

After these procedures, and at the end of the experiment, all the 10 animals of each group were sacrificed, and their urinary bladders were removed for collection of tissue fragments, which were embedded in paraffin and prepared for histological section, by the usual techniques, previously described in the present report.

The comparative results of the use of the compound of the invention used in combination or association with other compounds and compared to the isolated results for each compound are shown in Table 6-A.

The criteria for assessment of lesions, effects of treatment and assessment of toxic effects (hematuria) have been previously described in this specification.

TABLE 6-A

| | Group A | | | | | | | | |
|---|---|---|---|---|---|---|---|---|---|
| | n = 10 | Group B | | Group D | Group E | | | | Group I |
| | Untreated | n = 10 | Group C | n = 10 | n = 10 | | | | N = 10 |
| | control + | MNU + | n = 10 | BCG + | Mitomycin C + | Group F | Group G | Group H | Valrubicin + |
| Histological | saline | saline | Polymeric | Polymeric | Polymeric | n = 10 | n = 10 | N = 10 | Polymeric |
| assessment | 0.9% | 0.9% | anhydride | anhydride | anhydride | BCG | Mitomycin C | Valrubicin | anhydride |
| Flat hyperplasia | — | — | 4 (40%) | 4 (40%) | 3 (30%) | 2 (20%) | 1 (10%) | 1 (10%) | 40 (%) |
| Papillary | — | — | 3 (30%) | 3 (30%) | 4 (20%) | 2 (20%) | | 1 (10%) | 4 (40%) |
| hyperplasia | | | | | | | | | |
| Low-grade | — | 4 (40%) | | 1 (10%) | 1 (20%) | 3 (30%) | 6 (60%) | 4 (40%) | 1 (10%) |
| intraepithelial | | | | | | | | | |
| carcinoma | | | | | | | | | |
| High-grade | — | 5 (50%) | | | | 2 (20%) | 3 (30%) | 2 (20%) | |
| intraepithelial | | | | | | | | | |
| carcinoma | | | | | | | | | |
| (carcinoma in | | | | | | | | | |
| situ-Tis) | | | | | | | | | |
| Papillary | — | 1 (10%) | | | | | | 1 (10%) | |
| carcinoma (Ta) | | | | | | | | | |
| Squamous | — | — | | | | 1 (10%) | | 1 (10%) | |
| metaplasia | | | | | | | | | |
| Normal | 100% | 0% | 3 (30%) | 2 (20%) | 2 (20%) | 0% | 0% | — | 1 (10%) |
| Toxicology: | Negative | — | Negative | Positive | Positive | Positive | Positive | Positive | Negative |
| Presence of | (0%) | | (0%) | n = 2 | n = 2 | n = 10 | n = 9 | N = 10 | |
| hematuria | | | | (20%) | (20%) | (100%) | (90%) | 100% | |

### Table 6-A—Discussion of the Results

Analysis of data shown in Table-6-A reveals that the topical use of the invention in intravesical instillation associated with other drugs (Mitomycin C, Valrubicin and BCG vaccine) not only showed synergistic effects (Group D, Group E, Group I—Table 6-A), or else, maximized the therapeutic effects, but also significantly reduced unwanted side effects (hematuria) compared to the isolated use (Groups F, G, H—Table 6-A) of the same compounds (BCG, Mitomycin C, Valrubicin).

Based on the data shown in Table 6-A, any person skilled in the art easily understands that the invention can replace the above mentioned drugs (BCG, Mitomycin C, Valrubicin) in the case of failure or occurrence of side effects that prevent their use.

It can be also seen that the use of the invention has notably contributed to reduce local toxicity (hematuria) when combined with Mitomycin C and BCG vaccine (Group D and Group E—Table 6-A) and also with Valrubicin (Group I—Table 6-A).

Also, information on the practical use of the invention, notably synergy with other drugs, i.e. maximization of therapeutic effects associated with the reduced toxicity of the combination (Group D, Group E, Group I—Table 6-A), clearly demonstrates the benefits of its use in clinical practice, i.e., in protocols combined with chemotherapy and immunotherapy drugs in the topical treatment of cancer affecting the inside and outside epithelium (Table-6-A—Group D, Group E, Group I) with the purpose of maximizing the effectiveness of the combined therapy and simultaneously minimizing or neutralizing inflammatory disorders, such as hematuria associated to the cancerous process and also with the toxicity of the drugs.

These data concerning the practical use of the invention, notably synergy, demonstrated by the maximization of the therapeutic effects when associated to other drugs, including reduction in toxicity, allow to forecast its use in clinical practice for the palliative treatment of cancer of the epithelial tissue lining inside or outside surfaces of the body, particularly in the urinary bladder, bowel, esophagus and uterus, when surgical treatments are no feasible due to medical problems related to high probability of unacceptable morbidity and/or high risk of mortality (Table 6-A).

### Formulations for the Invention in the Treatment of Epithelial Cancer

As already cited, in the current state of the art, the best therapy for tumors of the epithelial tissue is based on surgical removal of the lesions, when feasible, followed by immediate topical application of chemotherapy or immunotherapy drugs.

For the successful deposition or use of these compounds, it is necessary to develop pharmaceutical formulations capable of ensuring their solubilization and facilitating their adherence or concentration on cancerous tissues.

For the purposes of the present invention, the immunomodulator (proteic aggregate of ammonium and magnesium phospholinoleate-palmitoleate anhydride) was used in the form of aqueous suspension, a sterile solution of sodium chloride 0.9% in water (NaCl 0, 9%) in experiments of the practical use of topical treatment of cancer in the lining epithelium (urinary bladder), as described in Tables 4, 5 and 6-A.

As described in the present report, in the treatment of male patients, the immunomodulator (proteic aggregate of ammonium and magnesium phospholinoleate-palmitoleate anhydride) was also used in the form of a topical cream with 15% concentration of the active ingredient, using sterile vaseline and sterile deionized distilled water as pharmaceutical carriers (Table 7). For the treatment of premalignant lesions in female patients, the immunomodulator (proteic aggregate of ammonium and magnesium phospholinoleate-palmitoleate anhydride) was formulated using a topical cream with 20% concentration of the active ingredient (Table 8-B).

For all these formulations, either using saline solution (NaCl 0.9%) used in the experiments shown in Table 4, Table 5 and Table 6-A and also creams for topical application used in the experiments shown in Table 7, Table 8A and Table 8B, the invention was able to effectively fight malignant and premalignant lesions in the epithelial tissue lining surfaces inside the body (urinary bladder, uterus) and outside (skin) at the referred doses.

However, the immunomodulator (proteic aggregate of ammonium and magnesium phospholinoleate-palmitoleate anhydride) is a poorly soluble drug. Thus, in order to increase its solubility and improve its penetration in the epithelium, others pharmaceutically acceptable carriers were selected and tested in combination with the immunomodulator, including polymers, polyethylene glycols, polyethylene oxide (PEO), non-ionic surfactants and edible oils.

Furthermore, in the state of art, some polymers such polyoxyethylene-polyoxypropylene block polymers (U.S. Pat. No. 3,740,421 A) also named Poloxamers (Pluronics) are reported as able to induce functional alterations in cells.

Also, Poloxamers have been reported as able to preferentially target cancer cells, due to differences in the membrane of these cells when compared to noncancerous cells. For references: Batrakova et al. Pluronic block copolymers: Evolution of drug delivery concept from inert nanocarriers to biological response Journal of Controlled Release 130 (2008) 98-106doi:10.1016/j.jconrel.2008.04.013.

As a result, the Poloxamers (Pluronics) may cause important biological effects such as sensitization of tumors to various anticancer agents. For references: Batrakova et al. Pluronic block copolymers: Evolution of drug delivery concept from inert nanocarriers to biological response. Journal of Controlled Release 130 (2008) 98.

Finally, Poloxamers are reported as useful in the formulation of topically applicable cosmetic and pharmaceutical compositions in the closest state of art. For reference: U.S. Pat. No. 7,737,181 B2.

For all the reasons above cited, a Poloxamer (polyethylene-polypropylene-glycol (CAS registry number 9003-11-6) Molecular formula: HO. (C2H4O) m.(C3H6) n.H, Pluronic F68) was specifically selected to be tested as a possible pharmaceutical carrier, for the immunomodulator (proteic aggregate of ammonium and magnesium phospholinoleate palmitoleate anhydride) in an animal model for the study of urinary bladder cancer (Table 6-B).

The results obtained in animal model with the use of the immunomodulator (proteic aggregate of ammonium and magnesium phospholinoleate-palmitoleate anhydride) with its formulation, using polyethylene-polypropylene-glycol (Poloxamer), used as pharmaceutical carrier, when compared to the application of the immunomodulator (proteic aggregate of ammonium and magnesium phospholinoleate-palmitoleate anhydride) using saline solution (NaCl 0.9%) as pharmaceutical carrier were absolutely outstanding and unprecedented in the state of the art.

As shown in Table 6-B with a formulation using polyethylene-polypropylene-glycol (Poloxamer) as pharmaceutical carrier, for the same therapeutic effect in urinary bladder cancer, a 10 times lower amount of the active ingredient (proteic aggregate of ammonium and magnesium phospholinoleate-palmitoleate anhydride) was required (Table 6B—Group E).

Example of Practice Use for Comparative Purposes, Using Compound of the Invention in Saline Solution, and with Addition in Polymer (Poloxamer)—Table 6-B

Fifth (50) Fischer 344 rats, aged 7 weeks, weighing in average 150 grams were used in this experiment.

All the animals (Fischer 344 rats), except for the untreated control group (Table 6 B—Group A), were subjected to applications of 1.5 mg/kg of n-methyl-n-nitrosourea (MNU) dissolved in 0.30 ml of sodium citrate, intravesically, using a catheter (for insertion in the bladder) for 7 weeks for bladder cancer induction. After administration of MNU the animals were divided into 5 groups of 10 animals each.

One group (untreated control) of 10 animals was given only saline solution (NaCl 0.9%) also intravesically and for an equal period (Table 6 B—Group A). One group equally induced with MNU (Table 6-B Group B) did not receive any treatment for comparison purposes and assessment of lesions.

After the period of cancer induction the animals were treated with the compounds proteic aggregate of ammonium and magnesium phospholinoleate-palmitoleate anhydride at the dose of 10 mg/kg (Table 6 B—Group C) added to saline solution (NaCl 0.9%), the compound proteic aggregate of ammonium and magnesium phospholinoleate-palmitoleate anhydride at the final dose of 1 mg/ml mg/kg (Group-D) also added to saline solution (NaCl 0.9%).

Finally, after the period of cancer induction, the animals were also treated with the immunomodulator (proteic aggregate of ammonium and magnesium phospholinoleate-palmitoleate anhydride) at the dose of 0.2 mg dissolved in 0.2 mL of aqueous solution of Poloxamer (polyethylene-polypropylene-glycol) or else, at the final concentration of 1 mg/mL, (Table 6-B—Group E).

All the compounds were intravesically inoculated, with a catheter (for insertion into the bladder) for this purpose, for 6 consecutive weeks, for comparative assessment in the treatment of MNU-induced cancerous lesions.

The results are shown in Table 6-B below.

The comparative results of the use of the compound of the invention in saline solution (NaCl 0.9%) at the concentration of 10 mg/kg (Table 6 B—Group C), saline solution at the concentration of 1 mg/kg (Table 6 B—Group D) and finally of the immunomodulator (proteic aggregate of ammonium and magnesium phospholinoleate-palmitoleate anhydride) at the dose of 0.2 mg dissolved in 0.2 mL of aqueous solution of Poloxamer (polyethylene-polypropylene-glycol) or else, at final concentration of 1 mg/mL (Table 6-B—Group E) are shown in Table 6-B below.

The criteria used for assessment of the lesions, effects of treatment and assessment of toxicity (hematuria) for the experiments of Table 6-B have been previously described above in this specification.

TABLE 6-B
Histological assessment

| | | Group B | Group C | Group D | |
|---|---|---|---|---|---|
| | Group A | n = 10 | Immunomodulator + | Immunomodulator + | Group E |
| | n = 10 | MNU + | saline solution | saline solution | Immunomodulator + |
| | Untreated | saline | at 0.9% | at 0.9% | Poloxamer |
| | control | solution at | 10 mg/kg | 1 mg/ml | 1 mg/ml |
| | group | 0.9% | intravesical | intravesical | intravesical |
| Flat | — | — | 2 (20%) | — | 4 (40%) |
| hyperplasia | | | | | |
| Papillary | — | — | 6 (60%) | 1 (10%) | 2 (20%) |
| hyperplasia | | | | | |
| Low-grade | — | 1 (10%) | — | 2 (20%) | 1 (10%) |
| intraepithelial | | | | | |
| carcinoma | | | | | |
| High-grade | — | 3 (30%) | — | 1 (10%) | — |
| intraepithelial | | | | | |
| carcinoma | | | | | |
| (carcinoma in | | | | | |
| situ-Tis) | | | | | |
| Papillary | — | 4 (40%) | — | 1 (10%) | 1 (10%) |
| carcinoma (Ta) | | | | | |
| Squamous | — | 2 (10%) | 1 (10%) | 5 (50%) | — |
| metaplasia | | | | | |
| Normal | 100% | — | 1 (10%) | — | 2 (20%) |
| Toxicology: | 0% | 10 (100%) | 0% | 6 (60%) | 0% |
| Presence of | | | | | |
| hematuria | | | | | |

### Discussion of the Results

The therapeutical results for intravesical use of the immunomodulator (proteic aggregate of ammonium and magnesium phospholinoleate-palmitoleate anhydride) at the dosage of 10 mg/kg (Table 6 B—Group C), by intravesical instillation, in topical form, suspended in saline solution (NaCl 0.9%) are similar to those shown in Table 4 (Table 4—Group D).

Or else, with the topical use (10 mg/kg—Group C—Table 6-B) 90% of tumor reversion was obtained (Group C—Table 6-B) compared to the animals induced with MNU and treated only with saline solution (NaCl 0.9%), where malignant and premalignant lesions were found in all of them (100%) (Group B—Table 6-B).

The absolute novelty compared to the previous state is that the use of the immunomodulator (proteic aggregate of ammonium and magnesium phospholinoleate-palmitoleate anhydride) at the dose of 0.2 mg dissolved in 0.2 mL of aqueous solution of Poloxamer (polyethylene-polypropylene-glycol) or else, at final concentration of 1 mg/mL (Table 6-B—Group E) was equally effective in the treatment of cancerous lesions in the epithelium of the urinary bladder (Group-E—Table 6-B) with therapeutic results comparable to those of Group C—Table 6-B that used a dose (10 mg/kg) of the immunomodulator (proteic aggregate of ammonium and magnesium phospholinoleate-palmitoleate anhydride) 10 times greater to obtain a similar therapeutic effect.

The topical use of the immunomodulator (proteic aggregate of ammonium and magnesium phospholinoleate-palmitoleate anhydride (Group D—Table 6-B) at the dose of 1 mg/kg, dissolved in saline solution (NaCl 0.9%) did not show therapeutic effects compared to those observed in Group C—Table 6-B and in Group E—Table 6-B.

The experiment of the practical use of the invention demonstrates that the pharmaceutical formulation of the immunomodulator (proteic aggregate of ammonium and magnesium phospholinoleate-palmitoleate anhydride) with Poloxamer (polyethylene-polypropylene-glycol) was capable of producing unprecedented practical effects, measured by a remarkable decrease in the required amounts of the active ingredient (proteic aggregate of ammonium and magnesium phospholinoleate-palmitoleate anhydride) for the treatment of tumors in the epithelial tissue, in the specific case, those located in the urinary bladder.

Any person skilled in the art understands that a formulation that allows reducing the quantities of drugs required to obtain a therapeutic effect has undeniable economic advantages, and also in what concerns safety due to the smaller number of substances to be deposed in the body's tissue.

Therefore, the use of the Poloxamer (polyethylene-polypropylene-glycol) for the elaboration of formulations with the immunomodulator (proteic aggregate of ammonium and magnesium phospholinoleate-palmitoleate anhydride) represents a clear improvement in the state of the art, for the use of this compound in the situations in which it is required, as shown in the present invention.

### Cancer in the Epithelial Tissue Lining Surfaces Outside the Body—Skin Cancer—State of the Art

### Introduction

Like any other type of cancer, skin cancer is broadly defined as the uncontrolled growth of cells that have the potential to spread to or penetrate into other parts of the body, which usually occurs if such lesions are not detected and treated in a timely manner.

Skin cancer can be caused by many factors, one of the major factors being over exposure to UV radiation from the sun. It is considered the most common type of cancer, corresponding to 25% of all neoplastic lesions. For reference: http://www.cancer.gov/cancertopics/types/skin

Other causes include prolonged exposure to chemicals, particularly the action of some types of viruses such as the human papilloma virus or HPV, genetic factors and finally low immunity.

Regarding human papilloma viruses (HPV), these agents are listed as causes of premalignant cellular changes, also called cell dysplasias in the epithelial tissue lining surfaces inside or outside the body.

The papilloma viruses or human papilloma viruses (HPV) cause several clinical manifestations such as skin warts, genital warts and finally premalignant cell changes (dysplasias) in the epithelium lining the cervix and skin, which often can develop into cancer if left untreated.

In the state of the art, the treatments of lesions caused by HPV viruses in the epithelium lining surfaces outside the body (skin) involve elimination or surgical removal of the lesions, such as abrasion treatment, laser surgery and cryocauterization, for example.

However, the excision or elimination of the affected tissue alone is not is not able to prevent recurrence of the tumor or lesions caused by the virus in most occasions, and therefore the use of combined therapies is needed.

With the advance of the state of the art, some new drugs, notably immunomodulators such as Interferons, and the compound called Imiquimod, are now used as a topical adjuvant therapy against cutaneous forms of HPV associated to or preceded by previous surgical removal of the lesions.

In general, it can be said that the selection of the type of treatment of premalignant or malignant lesions will depend on the type of cancer, location of the cancer, patient's age and on whether the lesion is a primary cancer or a recurrence.

In the event of a cancer already manifested or diagnosed, in the state of the art, the typical treatment is surgical removal of lesions using different techniques such as abrasion treatment, laser surgery, cryocauterization, etc followed by radiotherapy or chemotherapy with topical action. For example, surgery followed by topical chemotherapy may be indicated for the treatment of superficial basal cell carcinoma.

For low-risk tumors, in some cases, external radiation or topical chemotherapy with compounds as imiquimod, resiquimod and the compound called 5-fluorouracil and other procedures such as cryotherapy may be suitable.

Other types of treatment such as photodynamic therapy, with compounds that activate with polarized light or laser can also be used in combination with surgical excision of the tumors or premalignant lesions.

Other more aggressive epithelial cancers such as melanoma, which is considered the most lethal type of skin cancer, are less sensitive to radiation or chemotherapy, but can be fought with a reasonable degree of success due to the combination of surgical excision of tumors followed by the application of topical or systemic immunotherapy, particularly if detection of malignant or premalignant lesions is made early.

In short, it is well established in the state of the art that the best treatment for malignant or premalignant lesions affecting the epithelial tissue lining outside the body (skin) should include a combination of therapies, particularly those with topical action, to maximize therapeutic effectiveness and minimize the occurrence of relapses.

The same understanding applies for malignant or premalignant lesions affecting the epithelial tissue lining surfaces inside the body, e.g. bladder cancer, esophagus cancer and premalignant or malignant uterine lesions caused by several types of HPV.

The present invention, due to the novel and unprecedented functionalities of the immunomodulator (proteic aggregate of ammonium and magnesium phospholinoleate-palmitoleate anhydride) presented in this report is especially suitable to meet these requirements and also represents a novel and remarkable advance in the state of the art for the topical treatment of malignant lesions in the epithelial tissue lining outside and inside surfaces of the body.

For illustration purposes, two examples of the practical use of the invention in the topical use of lesions associated to HPV virus affecting the epithelial tissue lining surfaces outside (skin) and inside the body (uterus) are provided.

These practical examples are merely illustrative and do not intend to reduce or limit the field of application or the scope of the present invention.

Besides indicating the effectiveness of the invention in the treatment of these specific diseases, the results obtained in these experiments of practical use also demonstrate the usefulness of the invention for other situations that may require topical treatment against malignant or premalignant lesions in the epithelial tissue lining outside or inside surfaces of the body.

### Example of Practical Use—Use of the Invention for the Topical Treatment of Genital Warts Associated to Virus (HPV)—Table-7

For this experiment of the practical use of the invention, 10 volunteers were selected, all of them male individuals, with warts around the penis, clinically diagnosed as condylomata acuminata also named genital warts.

Genital warts (condylomata acuminata) are clinical symptoms of a highly contagious sexually transmitted disease caused by some types of human papillomavirus (HPV). Genital wart occurs in human beings in the skin, in genital or anal area, as well as in the mucous membranes of the rectum, cervix and vagina.

It is very difficult to treat genital warts. Existing treatments are focused on the removal of visible warts, but these may also regress. The reason is that although treatments can remove the warts, but frequently they do not remove the HPV, so warts can recur after treatment. For reference: Buck H W. Warts (genital). Clin Evid (Online). 2010 Aug. 13; 2010. pii: 1602.

All patients were admitted for treatment with the compound of the invention showing recurrence of lesions after previous topical treatment of genital warts with podophyllin (podophyllotoxin).

Laboratory tests of tissue samples from the lesions of all patients, prior to the treatment, were analyzed by in situ hybridization technique and found to be positive for HPV type 11), confirming the clinical diagnosis of condylomata acuminata or genital warts of the penile region associated to HPV.

Of the 10 voluntary patients, 6 (patients 1, 2, 3, 4, 5 and 6) were subjected to excision of the genital warts with topical application of podophyllin (podophyllotoxin—CAS number: 518-28-IUPAC:(10R,11R,15R,16R)-16-hydroxy-10-(3,4,5-trimethoxyphenyl)-4,6,13-trioxatetracyclo[7.7.0.03, 7.011,15]hexadeca-1,3(7),8-trien-12-one) in the affected regions for 4 days, with an interval of 2 days, followed by daily topical application for each patient of the compound of the present invention (proteic aggregate of ammonium and magnesium phospholinoleate-palmitoleate anhydride) suspended in appropriate vehicle (cream for topical application—pH 7) composed of sterile vaseline and deionized sterile distilled water with 15% concentration of the active ingredient (proteic aggregate of ammonium and magnesium phospholinoleate-palmitoleate anhydride) for 4 more days.

Of the 10 patients, 4 (patients 7, 8, 9, 10) were subjected only to the previous treatment, i.e., excision of genital warts with topical application of podophyllin (podophyllotoxin) for 8 days in total, without the subsequent administration of the compound of the present invention.

The participants were monitored for 30 more days for assessment of possible recurrence of lesions and again subjected to laboratory analysis with in situ hybridization technique to evaluate the possible presence of HPV.

### Results

Remarkable results were obtained with the topical use of the compound of the present invention. Of the patients who used the combined treatment (patients 1, 2, 3, 4, 5, 6), only one patient (Table 7—patient 2) showed recurrence of lesions, i.e. new reappearance of lesions (warts or condyloma) in the penile region.

Post-treatment laboratory test (in situ hybridization) of this patient (patient 2) was also positive for the presence of HPV.

All the other patients (patients 1, 3, 4, 5, 6), did not show recurrence of lesions (warts or condyloma) in a post-treatment clinical and laboratory follow-up.

The patients without recurrence who used the compound of the present invention (patients 1, 3, 4, 5, 6) were also negative for the presence of HPV when subjected to specific laboratory test (in situ hybridization).

All the patients who were given only Podophyllin (patients 7, 8, 9, 10) were found to be positive for a new recurrence of genital warts associated with the residual presence of HPV virus detected in specific laboratory test (in situ hybridization).

TABLE 7

| | | | | Application of | | |
|---|---|---|---|---|---|---|
| | | | | Podophyllin (4 | | Post- |
| | Clinical | Pre-treatment | Application | days) + | Clinical | treatment in |
| | presentation: | in situ | of | immunomodulator | presentation:: | situ |
| | Relapsed | hybridization | Podophyllin | in topical form (4 | Recurrent | hybridization |
| Patients | condyloma | for HPV | (8 days) | days) + (4 days) | condyloma | for HPV |
| Patient 1 | positive | positive | no | yes | negative | negative |
| Patient 2 | positive | positive | no | yes | positive * | positive* |
| Patient 3 | positive | positive | no | yes | negative | negative |
| Patient 4 | positive | positive | no | yes | negative | negative |
| Patient 5 | positive | positive | no | yes | negative | negative |
| Patient 6 | positive | positive | no | yes | negative | negative |
| Patient 7 | positive | positive | yes | no | positive | positive |
| Patient 8 | positive | positive | yes | no | positive | positive |
| Patient 9 | positive | positive | yes | no | positive | positive |
| Patient 10 | positive | positive | yes | no | positive | positive |

### Discussion of the Results—Table 7

It can be concluded that the use of the compound of the present invention, topically applied to the epithelium, showed a clinical and laboratory efficacy of approximately 90% (Table-7), which undoubtedly represents a remarkable gain in effectiveness, compared to one of the main types of treatment in the state of the art, for the topical treatment of this type of lesion associated to the presence and action of HPV virus.

Although in this case the procedure used was removal of the genital warts, after excision of the lesions by chemical means, i.e. with the use of podophyllin (podophyllotoxin), the invention can also be used in combination and association with other therapies or techniques, as, for example, removal by cryocauterization or else electrocauterization and other topical surgical procedures.

In conclusion, this experiment of practical use demonstrates that the invention can be advantageously used as adjuvant therapy for the treatment for premalignant lesions, notably those caused by aggressive agents, e.g. genital warts related to HPV in the epithelial tissue lining outside surfaces, associated to other existing procedures in the state of the art, such as surgical procedures and others.

### Premalignant Lesions of the Uterine Cervix and Evolution to Cancer—Associated Factors—Treatments—State of the Art

The so-called cervical dysplasia is considered a premalignant or precancerous change in epithelial cells of the cervix. For illustrative purposes, we cite: Kevin A. Ault—Epidemiology and natural history of human papillomavirus infections in the female genital tract. Infect Dis Obstet Gynecol. 2006; 2006 Suppl: 40470. Infect Dis Obstet Gynecol. 2006; 2006:40470.

In some cases, cervical dysplasia remains stable or is eliminated or reversed by the immune system. However, if not timely treated, it may become a chronic condition and progress to cervical cancer.

Cervical dysplasia is routinely diagnosed in clinical practice with the Papanicolaou test, also named Pap-smear test, which is used for early detection of the lesions.

The so-called Pap-smear test is a routine gynecological test with cells collected from the cervical region (cells from the uterine cervix). It shows signs related to infections in the region, as well as malignant lesions.

Besides its classical use for assessment of cells of the cervix, the test is also used to evaluate the effect of treatments.

The result of Pap smears is grouped into 5 (five) classes: Class I means absence of abnormal cells, Class II generally indicates inflammation or infection and Class III indicates the presence of abnormal cells (dysplasia).

Class III can be subdivided into three subclasses or types of dysplasia: mild, moderate or severe.

Cauterization is usually indicated in mild and moderate dysplasia. In severe dysplasia, a surgery to remove a cone-shaped piece of tissue from the cervix (conization) or other surgical measures may be needed.

Class IV dysplasia is a highly suspicious finding for malignancy and Class V indicates manifestation of cervical cancer.

### Determinant Causes for Cervical Dysplasia—Chronic Infection by HPV

It is well established in the state of the art that the determining factor for most chronic cervical dysplasias, which if not effectively treated may lead to cervical cancer, is the infection caused by the sexually transmitted Human Papilloma virus—HPV.

There are approximately 100 types of identified HPV, of which about a dozen are involved in the process that generates cervical dysplasia and its progression to cervical cancer. HPV-16 and HPV-18 types are responsible for 60% of the cases of cervical cancer worldwide. Munoz N, Castellsague X, de Gonzalez A B, Gissmann L. Chapter 1: HPV in the etiology of human cancer. Vaccine. 2006; 24 Suppl 3:S1-S10. See also: Rositch A F et al. The incidence of human papillomavirus infection following treatment for cervical neoplasia: a systematic review. Gynecol Oncol. 2014 March; 132(3):767-79.

HPV 6, HPV-11, HPV-16, HPV-18, HPV-33 and HPV-45 are high-risk/carcinogenic HPV types. For an example of the state of art, we cite: Rositch A F et al. The incidence of human papillomavirus infection following treatment for cervical neoplasia: a systematic review. Gynecol Oncol. 2014 March; 132(3):767-79.

These types cited (HPV 6, HPV-11, HPV-16, HPV-18, HPV-33, HPV-45) and recognized as being of high risk are associated to the development of cervical dysplasias, i.e. precancerous cell lesions, as they may progress to cancer. The presence and characterization of these viral types are assessed in the state of the art, following diagnosis of cervical dysplasia through several laboratory techniques such as in situ hybridization and PCR (Polymerase chain reaction).

There is no evidence yet in the state of the art of whether cell changes called dysplasias (cervical dysplasia) may be directly associated to viral activity (direct cytopathogenic effect) or indirectly associated due to cell changes induced by host-pathogen interaction (indirect cytopathogenic effect) or a combination of these causes.

However, regardless of the causal factors of cervical dysplasia, any expert with knowledge of the state of the art can easily understand that the concomitant treatment of the primary carcinogenic cause, that is, chronic viral infection, prior to, associated with or subsequent to treatment or removal of dysplasia, which is a lesion usually associated to the presence of HPV, is important for a successful therapy because it reduces the chance of recurrence of the infection that can lead to dysplasia again.

A hypothetical ideal treatment of a precancerous condition or cellular change, that is, cervical dysplasia, which is associated to chronic infection by HPV, should attack or remove abnormal cells or cause lesions to regress to less aggressive classes according to the Papanicolaou (Pap) classification, and at the same time contribute to the control of the initial causative agent, that is, HPV. As will be shown in the present report, this can be obtained with the present invention.

### Cervical Dysplasia—Preventive Therapies—Options of Treatment of Causal Agents (HPV)

Since it has been proven that chronic infection by HPV may induce dysplasias and progression of this condition to cancer, various treatments were developed and are available in the state of the art to attempt to control and/or eliminate the viruses and associated lesions and/or at least to provide a palliative treatment of the dysplasias and cancer. For examples of the state of the art: Scheinfeld N, Lehman D S. An evidence-based review of medical and surgical treatments of genital warts. Dermatol Online J. 2006 Mar. 30; 12(3):5. Retrieved from: http://escholarship.org/uc/item/7v57p744

Infection by HPV can occur in three main presentation forms: clinical, subclinical and latent: The clinical form, with the presence of macroscopic lesions (genital warts) in the anogenital region; the subclinical form, characterized by the presence of diffuse epithelial hyperplasia and dysplasia, seen through the colposcope and after application of contrast medium (acetic acid) and the latent form, that is, without histological changes, though with the presence of the viral DNA detected by techniques such as hybridization, hybrid capture or PCR (polymerase chain reaction).

The traditional treatments aimed mostly to the elimination of lesions associated to HPV or its clinical manifestations, such as genital warts, involve topical therapies, with the use of corrosive agents such as podophyllin and its derivatives, such as podophyllotoxin and also trichloroacetic acid. Such treatments are often toxic and painful. For reference: Geo von Krogh, Eric Longstaff. Podophyllin office therapy against condyloma should be abandoned. Sex Transm Infect 2001; 77:409-412 doi:10.1136/sti.77.6.409

Several surgical techniques are also used with varying degrees of success in the state of the art: local excision, cryotherapy, CO2 laser vaporization and electrocauterization. However, in the state of art, also the surgical treatments for the clinical and subclinical forms of HPV are not very efficient and have a high degree of recurrence. They involve long periods of time and are often painful and/or disfiguring.

### Problems Involving Surgical Treatments for the Elimination of Dysplasias

Surgical procedures to remove HPV infected tissues can be painful and impractical for the treatment of extensive lesions. In the state of the art there is great controversy as to whether the frequent post-treatment recurrences are due to reactivation of subclinical infection or derived from normal epithelium left untreated. For examples of the state of the art, we cite: Krebs, H.-B., 1989. Management strategies. Clinical Obstetrics and Gynecology. 32(1): 200-213 and Leszczyszyn J et al. Anal warts (condylomata acuminata)—current issues and treatment modalities. Adv Clin Exp Med. 2014 March-April; 23(2):307-11.

The most frequent complications include pain, local secretion, ulceration, infection, delayed healing. Permanent scarring and risk of subfertility may also occur. For examples of the state of the art, we cite: Spracklen CN1, Harland K K et al. Cervical surgery for cervical intraepithelial neoplasia and prolonged time to conception of a live birth: a case-control study. BJOG. 2013 July; 120(8):960-5.

### Recent Advances in the State of the Art—Immunotherapy

In the state of the art, the use of immunotherapy for the treatment of HPV infection and the damage caused by this virus to the cells (dysplasias) started with exogenous interferons (e.g. Interferon-alpha, interferon-beta, interferon-gamma), topically or intralesionally applied, to increase immunity. When used for the treatment of genital warts associated with HPV, locally-used interferon appears to be more effective than systemically-used interferon, For example of the state of the art, we cite: Yang J et al. Interferon for the treatment of genital warts: a systematic review. BMC Infect Dis. 2009 Sep. 21; 9:156.

With the development of new compounds, the immunotherapy alone or associated with other treatments becomes an attractive therapeutic option in the state of the art for the treatment of HPV infections. Among the new compounds available in the state of the art, we cite the immunomodulator called Imiquimod: (IUPAC: -(2-methylpropyl)-3,5,8-triazatricyclo[7.4.0.02,6]trideca-1(9), 2(6), 4,7,10,12-hexaen-7-amine). For reference: Scheinfeld N. Update on the treatment of genital warts. Dermatol. Online J. 2013 Jun. 15; 19(6):18559.

### Advances in the State of the Art—Prophylactic and Therapeutic Vaccines Against HPV

The most recent advances in the state of the art concern the development of vaccines against HPV, which act in a preventive way against the main carcinogenic types of HPV (HPV-6, HPV-11, HPV-16 and HPV-18). They are only efficient for women who have not yet reached sexual maturity or with no prior infection with HPV. There is not yet a therapeutic vaccine against HPV that ensures its elimination in patients already infected or with a previous history of infection. For reference: Kuang S K et al. Current status of human papillomavirus vaccines. Clin Exp Vaccine Res. July 2014; 3(2): 168-175. See also: Anne S., Rachel S. et al—Efficacy of the HPV-16/18 AS04-Adjuvanted Vaccine against Low-Risk HPV Types (PATRICIA Randomized Trial): An Unexpected Observation. J Infect Dis. (2013) 208 (9): 1391-1396.

### Treatment of Precancerous Lesions and Cervical Dysplasias—State of the Art

In the state of the art, it is widely accepted that the first stage of development of cervical cancer in women is the condition known as dysplasia, which occurs when squamous cells in the cervical region become abnormal in size and shape and begin to multiply. For reference: Doug L. Human papillomavirus, cervical cancer prevention, and more. Vaccine, Volume 26, Supplement 10, 19 Aug. 2008, Pages iii-iv

The condition named cervical dysplasia (Papanicolaou Grade III) is classified as mild, moderate or severe, depending on the degree of cellular abnormality on microscopic examination. As cited, usually cellular abnormalities are detected and classified by means of a procedure named Pap smears test (Papanicolaou test). For reference: Doug L. Human papillomavirus, cervical cancer prevention, and more. Vaccine, Volume 26, Supplement 10, 19 Aug. 2008, Pages iii-iv

In mild dysplasia, abnormal cells appear only on the surface layer of the epithelium. This condition may progress if not timely treated, evolving to severe dysplasia.

If not treated, severe dysplasia will progress in most cases to the maximum stage or degree, which is early cervical cancer, also known as in situ carcinoma (Tis-TNM classification).

Surgical procedures performed by mechanical, electrical (cauterization) and freezing (cryosurgery) are often used to eliminate and/or remove cervical epithelial lesions usually associated to the action of the human papilloma virus (HPV). Chemical abrasion with the use of corrosive chemicals, such as Podophyllin and its derivatives are mainly indicated for treatment of epithelial external lesions, because it's high toxicity.

Regardless of the techniques used to remove tissue damaged or changed, it is evident for any expert with knowledge of the state of the art that the success of preventive, curative or palliative treatments of precancerous lesions, that is, cervical dysplasias of any nature, to avoid progression to cancer, is also influenced by the efficient treatment of the main causative agent, that is, HPV, particularly the carcinogenic types (HPV 6/11/16/18).

### Treatment of Precancerous Lesions and Cervical Dysplasias—Current Problems—State of the Art

In the state of the art, recurrence rates for all the current excision or surgical treatments used to eliminate/treat precancerous cervical lesions or alterations (dysplasias) are very high, precisely because of the great difficulty in eliminating the main causative agent, that is, the human papilloma virus or HPV.

Given these facts and the remarkable and unprecedented properties of the immunomodulator (proteic aggregate of ammonium and magnesium phospholinoleate-palmitoleate anhydride) demonstrated in the topical use of the compound in the treatment of urinary bladder cancer (Table 4, 5, 6-A, 6-B) and also in the treatment of lesions in the external epithelium, caused by the HPV virus (Table 7), the invention was also tested for topical treatment of cervical dysplasia in women, being associated to the presence of the human papilloma virus (HPV), resulting in a new and unprecedented use for the compound, i.e., the intrauterine topical application of the immunomodulator (proteic aggregate of ammonium and magnesium phospholinoleate-palmitoleate anhydride) for the treatment of the referred disease.

Therefore, in order to demonstrate in practice the effectiveness of the present invention in the treatment of HPV-related cervical dysplasia, an example will be provided of its use in a clinical trial involving patients with chronic HPV infection and cervical dysplasias associated to the infection ranging from mild to moderate, which did not respond satisfactorily to one of the most common standard treatments in the state of the art, that is, a surgical procedure called electrocauterization.

### Practical Example of the Use of the Invention for the Topical Treatment of Cervical Lesions Associated to Virus (HPV)—Table 8-A and Table 8-B

In order to demonstrate in practice the effectiveness of the present invention in the topical treatment of HPV-related cervical dysplasia, an example of its use in a clinical trial involving patients with chronic and recurrent HPV infection and cervical dysplasias associated to the infection, ranging from mild to severe, which did not respond satisfactorily to one of the most common standard treatments in the state of the art, that is, electrocauterization surgery

This practical example is included for illustrative purposes only without intending to limit the field of application or the scope of the invention.

Clinical study: Postsurgical application of the immunomodulator (proteic aggregate of ammonium and magnesium phospholinoleate-palmitoleate anhydride) in 25 women with moderate to severe dysplasia confirmed by laboratory tests (Papanicolaou test) and HPV seropositivity (PCR method).

All patients had previous history of routine surgical procedures for removal of the damaged tissue (electrocauterization) in the months prior to the clinical trial, with recurrence of dysplasia and infection in all the cases.

The patients underwent routine examinations before the clinical trial for evaluation of the degree of dysplasia (Papanicolaou test) and cervical smears were taken for human papilloma virus (HPV) testing and typing.

Of the 25 patients that participated in the clinical trial, 15 were classified as suffering from moderate cervical dysplasia associated to HPV 11, 5 with moderate cervical dysplasia associated to HPV 18, and 5 with severe cervical dysplasia associated to types HPV-16 and HPV-18.

The results of the tests of patients on admission to the trial are shown in Table 8-A.

TABLE 8-A
Classification of the degree of cervical dysplasia and viral typing
performed on admission to the clinical trial (basal)

| Number of | Mild | Moderate | Severe | |
|---|---|---|---|---|
| patients | dysplasia | dysplasia | dysplasia | Type of HPV |
| n = 25 | (**) | (**) | (**) | associated (*) |
| 15 | — | 15 | — | HPV 11 |
| 5 | — | 5 | | HPV 18 |
| 5 | — | — | 5 | HPV 16/18 |

(*) Method for detection and viral typing: PCR - Kit Amplicor (HPV) - Roche
(**) Method for detection and classification of cervical dysplasia: Papanicolaou

Periodicity: Collection of cervical smear for the test and classification of dysplasia and HPV were performed in the pretreatment period (basal) and 6 months after the end of the clinical trial.
Characterization of the sample: Patients previously treated with surgery (electrocauterization) and showing signs of recurrent cervical dysplasia, classified by the Papanicolaou test as moderate to severe dysplasias, upon admission to the clinical trial (Table 8-A).
Viral typing: All patients (100%) had at least one viral type considered of high risk for developing malignancy on admission, and five patients showed association of more than one viral type (HPV 16 and HPV 18) (Table 8-A).
Clinical trial design: All patients underwent the same surgical procedure, that is, electrocauterization for removal of the abnormal tissue (dysplasia).

The patients were also given the (proteic aggregate of ammonium and magnesium phospholinoleate-palmitoleate anhydride), topically administered, for this particular purpose in the form of a topical cream (pH 7.0), composed of sterile vaseline and deionized sterile distilled water with a concentration of the active ingredient (proteic aggregate of ammonium and magnesium phospholinoleate-palmitoleate anhydride) of 20% daily applied in the uterus (cervix) using an appropriate applicator for 2 weeks, the first dose beginning 1 day after surgical cauterization (electrocauterization).

Follow-up: All patients were followed monthly through clinical examinations and colposcopy.
Final assessment: the patients were assessed again 180 days after the end of the surgical procedure combined with the applications of the immunomodulator (proteic aggregate of ammonium and magnesium phospholinoleate-palmitoleate anhydride), topically administered, for classification of the type of dysplasia and the possible presence of HPV.
Results: The results are shown in Table 8-B and then discussed.

TABLE 8-B
Classification of the degree of cervical dysplasia and viral
typing after the clinical trial (T: 80 days after the end)

| | Mild | Moderate | Severe | HPV type |
|---|---|---|---|---|
| Number of | dysplasia | dysplasia | dysplasia | associated |
| patients | (**) | (**) | (**) | (*) |
| 20 | — | — | — | — |
| 3 | — | | — | HPV 11 |
| 2 | — | — | | HPV 16/18 |

(*) Method for detection and viral typing: PCR - Kit Amplicor (HPV) - Roche
(**) Method for detection and classification of cervical dysplasia: Papanicolaou test

### Table 8-B—Discussion of the Results

The topical use of the compound of the present invention (proteic aggregate of ammonium and magnesium phospholinoleate-palmitoleate anhydride) associated to a surgical procedure (electrocauterization) showed remarkable results for the patients who participated in the clinical trial, both regarding cervical dysplasia and the causative agent (HPV).

Although the sample is limited in number, it should be noted that all patients had previously undergone the same surgical procedures for removal of the abnormal tissue (cervical dysplasia) and relapsed, that is, 100% of recurrence was observed.

The recurrence rate with the use of the invention, that is, the immunomodulator (proteic aggregate of ammonium and magnesium phospholinoleate palmitoleate anhydride) associated to surgery (electrocauterization) was 0% (zero percent), i.e. none of the patients showed any sign of dysplasia during the assessment performed 180 days after the end of the clinical trial, although 5 (five) patients still had HPV on the day of assessment after the end of the clinical trial, that is, 180 days later.

Comparatively, the rate of recurrence prior to the beginning of the experiment, reported for these patients, when treated only with surgical methods, was 100% of recurrence for cervical dysplasia and viral presence.

It should be noted that these patients no longer had cervical dysplasia in the assessment period (180 days after the end of the clinical trial), which clearly indicates a direct effect of the invention on cellular change (cervical dysplasia), that is, on cells with potentially precancerous lesions, regardless of the presence of the virus, since the laboratory tests indicated the presence of HPV in 5 (five) patients.

This effect of the invention on the precancerous lesion (cellular dysplasia), regardless of the presence of the virus, has been clearly demonstrated, since for all patients of the clinical trial who were diagnosed with dysplasia ranging from moderate to severe, the dysplasia was completely eliminated after treatment with the invention, despite de presence of the virus in a small percentage, that is, only 5 (five) patients (Table 8-B).

### Absolute Novelty of the Invention for the Topical Treatment of Precancerous Lesions Associated to HPV

The use of the immunomodulator (proteic aggregate of ammonium and magnesium phospholinoleate-palmitoleate anhydride) as a component of drug associations or combinations against facultative or strict intracellular pathogens or parasites is described in WO 2009/097670 A1, which includes general antiviral action, once these pathogens are classified as strict intracellular parasites.

However, it can be affirmed that this previous invention (WO2009/097670 A1), or else, the practical use of the immunomodulator (proteic aggregate of ammonium and magnesium phospholinoleate-palmitoleate anhydride) for the treatment of infections caused by intracellular parasites is totally different from the present invention, including the specific treatment of associated precancerous lesions or HPV-related cervical dysplasias.

The previous invention (WO 2009/097670 A1) concerns the use of the immunomodulator (proteic aggregate of ammonium and magnesium phospholinoleate-palmitoleate anhydride), systemically administered, that is, with the immunomodulator (proteic aggregate of ammonium and magnesium phospholinoleate-palmitoleate anhydride) injected into the body (intramuscular application) for the treatment of infectious diseases, including those of viral origin.

Finally, the use of the immunomodulator (proteic aggregate of ammonium and magnesium phospholinoleate-palmitoleate anhydride) as adjuvant therapy of cervical dysplasia and infection by HPV is described in WO 2011082458 A1. However, such practical use described in WO 2011082458 A1 concerns a different application, that is, the systemic use of the immunomodulator (proteic aggregate of ammonium and magnesium phospholinoleate palmitoleate anhydride), which must necessarily be introduced in the patient's body by intramuscular injection.

This means that in the previous requests, that is, in WO 2011082458A1 and also WO 2009/097670 A1 the described usefulness or practical effect of the invention depends on the use of parenteral route of administration of the compound (injection administration) to produce the desired systemic effects.

As for the present invention, it is intended for a completely different purpose, that is, the practical use of the immunomodulator (proteic aggregate of ammonium and magnesium phospholinoleate-palmitoleate anhydride), alone or associated to other non-drug and/or drug treatments also for the treatment of cancer, though by topical administration, that is, directly applied to the damaged tissue, which is completely different from the previous requests, providing several important practical benefits compared to systemic administration, which shall be described in this report.

### Comparative Advantages of the Use of Topically Administered Drugs

Any expert with knowledge in the state of the art can easily understand that the topical use, in the case of similar drugs, offers several and significant advantages compared to the systemic use, since the latter depends, for introducing substances into the body, on methods that cause their metabolism (e.g. oral), or else of potentially aggressive methods that cause poor patient compliance, such as the use of subcutaneous and/or intramuscular injections, for example.

Furthermore, as cited in the specification, therapies based on drugs that require use of enteral routes of administration (e.g.: systemic action through the GI tract) and also parenteral routes of administration (e.g.: systemic action, delivered by routes other than the GI tract) are not fully effective for the treatment of malignant or pre-malignant lesions in the superficial layer of the epithelial tissue lining surfaces outside or inside body organs.

For instance, the topical (intravesical) use of the immunomodulator (proteic aggregate of ammonium and magnesium phospholinoleate palmitoleate anhydride), for treatment of urinary bladder cancer in animals (Table 4—Group D) was more effective than the use of the same compound when used by subcutaneous injection (Table 4—Group E).

Others advantages of the use of topically administered drugs, including the invention, compared to injectable similar compounds, are cited in a non-exhaustive list, as follows:

A) A lower rate or possibility of occurrence of adverse or toxic effects, such as pain and/or muscle inflammation for instance and even less discomfort for patients who use the topical pharmaceutical form, which undeniably leads to greater patient adherence to treatment protocols;
B) Regarding similar drugs, another comparative advantage provided by the topical use is the absolute control of the amounts of drugs to be introduced into the patient's body. In other words, much larger amounts or concentrations of the active ingredient can be used at the site of the lesion compared to the amounts or concentrations of the same compounds that require systemic administration, for example, oral administration or injection;

Inversely, the topical administration of drugs, also allows the use of smaller amounts of the active ingredient at the site of the lesion, without loss of efficacy, when compared to the amounts or concentrations that can be used with the same compounds, though systemically introduced into the body (orally or by injection).

Or else, the use of drugs capable of topical action allows entire control of the amounts deposed in the body, which cannot be obtained with drugs that require systemic administration, e.g., orally or injected, which necessarily involves their metabolism by the body prior to distribution or deposition in the body tissues, including the damaged tissue.

C) Another comparative advantage of the topical use of drugs, including the invention, is the minimization or even elimination of the occurrence of chemical and/or structural changes in the drug caused by its metabolism in the body. This means that the maximum possible amount of the active ingredient can be used on the site of the lesion or disease, by topical administration. Finally, among other comparative advantages of the topical use of drugs, including the invention, also may be cited: it avoids fluctuation in drug levels due intra patient variations, improves the ability to deliver drug more selectively to a specific site, the avoidance of gastro-intestinal incompatibility, improves physiological and pharmacological response, and improves patient compliance.

### Comparative Advantages of the Use of the Invention in the Topical Treatment of Premalignant and Malignant Lesions of the Lining Epithelium.

Considering the information contained in the present report, and also examples of practical use, it can be affirmed that compared to the drugs used in the treatment of cancer affecting the epithelial tissue lining surfaces outside and inside the body, notably urinary bladder cancer, malignant lesion in the skin and uterine lesions associated to HPV, the present invention, when used alone and/or in association with other drugs and treatments known in the state of the art, allows significant advances over the state of the art.

These advances also include the previous use of the immunomodulator proteic aggregate of ammonium and magnesium phospholinoleate-palmitoleate anhydride described in WO 2009/097670 A1 and WO 2011082458 A1 where it is administered by parenteral via of administration such as subcutaneous, intramuscular and intraperitoneal.

Some benefits and advances allowed by the invention, that is, the topical use of the immunomodulator (proteic aggregate of ammonium and magnesium phospholinoleate-palmitoleate anhydride), considering the state of the art, follow:

A—It has been demonstrated, also with examples of practical use, that the topical administration of the compound of the present invention (proteic aggregate of ammonium and magnesium phospholinoleate-palmitoleate anhydride) has greater therapeutic effectiveness than the most effective immunotherapeutic drug in the closest state of the art for the topical treatment of urinary bladder cancer, which is BCG vaccine (Table 4, Table 6-A).
B—It has been shown, also with examples of practical use, that the compound (proteic aggregate of ammonium and magnesium phospholinoleate-palmitoleate anhydride), alone, in topical administration, has wider therapeutic effectiveness than the main chemotherapeutic agents in the state of the art for the treatment of malignant lesions of the epithelial tissue lining inside surfaces, notably in the urinary bladder, in topical application (Table-5)
C—The use of the invention, represented by the isolated use of the compound (proteic aggregate of ammonium and magnesium phospholinoleate-palmitoleate anhydride) in the treatment of urinary bladder cancer was not accompanied by side effects that are indicative of toxicity, such as hematuria (Table 4, Table 5, Table 6A, Table 6-B).
D) It has also been demonstrated, with examples of practical use, that the use of the invention in combination with chemotherapy and immunotherapy drugs (e.g. BCG), not only maximizes the effects of the compounds in the treatment of malignant lesions in the epithelial tissue lining inside surfaces, notably the urinary bladder, but also contributes significantly to the reduction of the unwanted side effects of such treatments (Table 5 and Table 6-A).
E) It has been demonstrated, also with examples of practical use of the invention, that the compound of the invention (proteic aggregate of ammonium and magnesium phospholinoleate-palmitoleate anhydride) can replace the treatments cited, that is, Mitomycin C, Valrubicin and BCG vaccine, in the case of failure or occurrence of toxicity that prevents their continued use (Table 5 and Table-6-A).
F) It has been demonstrated, also with examples of practical use, that the compound (proteic aggregate of ammonium and magnesium phospholinoleate-palmitoleate anhydride) used after chemical or surgical removal of lesions is effective as a topical adjuvant treatment of premalignant lesions of the epithelial tissue lining surfaces inside and outside the body associated to aggressive agents such as the HPV virus, notably in the skin and in the uterine region (Tables 7 and Table 8-B)

### Extrapolation of the Usefulness of the Invention for Tumors Located in Other Luminal Organs, Using Devices for the Application of Drugs

These remarkable effects of the use of the compound (proteic aggregate of ammonium and magnesium phospholinoleate-palmitoleate anhydride) in the treatment of cancer, by topical administration in the affected epithelium, illustrated in the examples cited in the present report, that is, topical treatment of precancerous lesions in the epithelium lining external surfaces (skin) associated to HPV demonstrate that the invention can be used for the topical treatment of malignant and premalignant lesions in the epithelial tissue lining outside (skin) and inside surfaces (mouth, esophagus, stomach, small intestine, urethra, urinary bladder, vagina, uterus).

Also, the remarkable effects of the use of the compound (proteic aggregate of ammonium and magnesium phospholinoleate-palmitoleate anhydride) in the treatment of cancer, topically administered to the affected epithelial tissue of inside surfaces, illustrated by examples cited in the present report, that is, topical treatment (intravesical) of urinary bladder cancer and of intrauterine lesions associated to HPV demonstrates the efficacy of the invention in the topical treatment of malignant and premalignant lesions in the epithelial tissue lining inside surfaces, notably in the urinary bladder and uterus.

The remarkable effects of the use of the compound (proteic aggregate of ammonium and magnesium phospholinoleate-palmitoleate anhydride) in the treatment of cancer, by topical application on the affected epithelium, illustrated by the experiments cited in the present report, i.e., the topical treatment (intravesical) of urinary bladder cancer, precancerous lesions in the epithelial tissue lining inside and outside surfaces indicate the use of the invention for the treatment of cancer or precancerous lesions affecting internal body regions, provided these can be reached by devices for administration of topical medication, such as catheters, cannulae and other instruments.

For the topical treatment of lesions in the epithelial tissue lining inside surfaces, all types of pharmaceutical vehicles known in the state of the art, such as creams, lotions and other vehicles of application and dispersion of drugs can be used for the purposes of topical application of the invention.

In short, all kinds of mechanical or pharmacological devices able to carry or release drugs can be used for the administration of the compound of the invention in the treatment of lesions in the epithelial tissue lining outside surfaces (skin) and also inside surfaces of luminal organs such as mouth, esophagus, stomach, intestines, urethra, urinary bladder, vagina, and uterus.

### Formulations or Pharmaceutical Forms for the Practical Use of the Invention

Three pharmaceutical formulations for the practical use of the invention that were found to be suitable for topical administration of the invention in animal models and in humans and were presented in this report follow.

The first formulation comprises essentially the suspension of the compound of the invention in sterile saline solution (NaCl 0.9%) for use or deposition in luminal organs for the treatment of premalignant or malignant lesions of the epithelial tissue lining inside surfaces, through catheters or other devices.

One advantage of this formulation is the possibility of its application on any epithelial tissue lining inside surfaces, such as mouth, esophagus, stomach, intestines, urethra, urinary bladder, vagina, and uterus, because saline solution at 0.9% (NaCl 0.9%) is known to be harmless to cellular components.

The second formulation which is also used in human subjects, mentioned in the present report (Table 7 and Table 8-B) comprises essentially the formulation of the proteic aggregate of ammonium and magnesium phospholinoleate-palmitoleate anhydride, with the concentration of the active ingredient ranging from 15% to 20-%, in the form of cream, which occurs when the compound is added and homogenized to a base or inert carrier, formed by sterile vaseline and sterile deionized distilled water, using a mixer. The final pH will be around 7.

The third formulation comprises the use of the compound solubilized with Poloxamer (Pluronics) with remarkable and outstanding practical results described in the present report and in Table 6-B.

For this formulation for topical use in the epithelial tissue lining inside or outside surfaces, 0.1 mg of the immunomodulator (proteic aggregate of ammonium and magnesium phospholinoleate-palmitoleate anhydride) is dissolved in 0.2 mL of aqueous solution of Ethylene Oxide/Propylene Oxide Block Copolymer (Poloxamer-Pluronic) with final concentration of 1 mg/ml.

This concentration (1 mg/ml) is the lowest concentration experimentally obtained to produce therapeutic effects with the topical use of the immunomodulator (proteic aggregate of ammonium and magnesium phospholinoleate-palmitoleate anhydride).

Below the concentration of 1 mg/ml for the immunomodulator (proteic aggregate of ammonium and magnesium phospholinoleate-palmitoleate anhydride) no therapeutic results were experimentally obtained, so far, in the topical treatment of lesions in the epithelial tissue lining inside and outside surfaces of the body.

### Additional Information on Future Uses of the Invention Alone or Associated to Other Drugs and Therapies

For the purposes of the present invention, the immunomodulator (proteic aggregate of ammonium and magnesium phospholinoleate-palmitoleate anhydride), can be used alone for topical application and also with other therapeutic modalities, in combination or association of treatments, also topically, as illustrated in the present report.

The other active substances for possible use with the invention, in association or combination with compounds, may vary in species, type, quality and quantity, according to the specific requirements of the disease.

The other active substances, with activity against the several types of malignant and premalignant lesions and also the causative agents, such as the HPV virus, are cited in the present report, only as specific examples of the different classes of products in the current state of the art, such as antineoplastic agents, chemotherapeutic and immunotherapeutic drugs, in a non-exhaustive classification or enumeration.

Other substances and therapies with topical action against cancer not specifically cited in the present report as known in the state of the art, or to be discovered and made available for usage according to the purposes of the present invention, can be used, as long as the immunomodulator (proteic aggregate of ammonium and magnesium phospholinoleate-palmitoleate anhydride) is used or maintained in the association or combination, for topical use, as widely described in this report, for it to continue to produce the desired effects.

Also, in some particular situations, as in the event of cancer affecting the epithelial tissue lining inside and outside surfaces, and also cancer located in other organs that are not part of the epithelial tissue, other substances and therapies active against cancer, with systemic action and possibly known in the state of the art, or to be discovered and made available, are also used or proposed for use in association or combined therapy, provided the use of the immunomodulator (proteic aggregate of ammonium and magnesium phospholinoleate-palmitoleate anhydride) is maintained in the association or combination, for topical administration, as widely described in this report, for the referred association or combination to continue to produce the desired effects in malignant and premalignant lesions in the epithelial tissue lining inside and outside surfaces.

### Compound Used for the Purposes of the Present Invention

The present invention uses a compound or immunomodulator known in the state of the art and called proteic aggregate of ammonium and magnesium phospholinoleate-palmitoleate anhydride, originally reported in PI-0305373-3 US 2006/0093628 A1, EP 1529784 A1, though used in the present invention in a way that is entirely different from all the previous applications of the product described so far in the state of the art, with the advantages associated to this specific form of use.

This compound now topically administered for the purposes of the present invention is characterized as proteic aggregate of ammonium and magnesium phospholinoleate-palmitoleate anhydride, with molecular weight 320.000 Dalton (320 KDa), being previously described in PI-0305373-3 US 2006/0093628 A1, EP 1529784 A1.

This compound used in the present invention, has shown in chemical analysis the presence of 11.6±4.0% of total lipids, 22.7±5.0% of palmitoleic acid, 42.9±2.0% of linoleic acid and 32.0±3.0% of oxidated linoleic acid, 20.1±0.9% of magnesium ions, 10.0±3.3% of ammonium ions, 45.2±2.7% of phosphate and 0.49±0.01% of proteins, according to PI-0305373-3 US 2006/0093628 A1, EP 1529784 A1.

The aminoacid distribution in the protein is: Asp 7.19%; Thr 3.56%; Ser 7.56%; Glu 8.53%; Pro 0.5%; Gly 9.69%; Ala 7.46%; Val 1.0%; Met 4.38%, Isoleu 2.54%, Leu 3.03%, Tyr 0.5%, Phe 1.0%, His 2.83%; Lys 3.56%, Trp 1.3% and Arg 35.2%, according to PI-0305373-3 US 2006/0093628 A1, EP 1529784 A1.

The method of production of the immunomodulator (proteic aggregate of ammonium and magnesium phospholinoleate-palmitoleate anhydride), used for the purposes of the present invention is equally contained and reported in the state of the art in PI-0305373-3 US 2006/0093628 A1, EP 1529784 A1, comprising the use of the Aspergillus oryzae fungus (A. oryzae) in appropriate culture medium, resulting in the immunomodulator specifically selected to be used for the purposes of the present invention.

For additional information related to access to the compound used in the present invention, mode of use, biological properties and other relevant information, please consult PI-0305373-3 US 2006/0093628 A1, EP 1529784 A1.

Without the need for additional information, any person skilled in the art, based only on information and explanations contained in the present report, can understand and use the invention to its full extent.

Although the invention is intended for human use, other animal species can benefit from its therapeutic properties.

We claim:

1. A compound for treating superficial cancer tumors located in epithelial tissue internal and external, used alone or comprising in combination:
(a) an immunomodulator, wherein the immunomodulator is a proteic aggregate of ammonium and magnesium phospholinoleate-palmitoleate anhydride, with molecular weight of 320.000 Dalton, having of 11.6±4.0% of total lipids, 22.7±5.0% of palmitoleic acid, 42.9±2.0% of linoleic acid, 32.0±3.0% of oxidated linoleic acid, 20.1±0.9% of magnesium ions, 10.0±3.3% of ammonium ions, 45.2±2.7% of phosphate, and 0.49±0.01% of proteins, and
(b) at least one anti-cancer agent suitable for treating the cancer tumors, said agent providing synergistic effects without additional toxicity when used with the immunomodulator.

2. The compound according to claim 1, wherein the at least one anti-cancer agent is selected from the group consisting of: Bacillus Calmette-Guérin (BCG), mitomycin, valrubicin, thiotepa, doxorubicin, cisplatin and combinations thereof.

3. The compound according to claim 1, wherein the amino acid content in the proteic aggregate is: Asp 7.19%, Thr 3.56%, Ser 7.56%, Glu 8.53%, Pro 0.5%, Gly 9.69%, Ala 7.46%, Val 1.0%, Met 4.38%, Isoleu 2.54%, Leu 3.03%, Tyr 0.5%, Phe 1.0%, His 2.83%, Lys 3.56%, Tip 1.3%, and Arg 35.2%.

4. A method of treating cancer affecting superficial internal epithelial tissues and superficial external epithelial tissues the method comprising administering a therapeutically effective amount of the compound of claim 1 to an organism, wherein the anti-cancer agent is suitable for treating the target infection.

5. The method according to claim 4, wherein the immunomodulator and anti-cancer agents are administered to the infected host organism either jointly, simultaneously, consecutively or sequentially.

6. A pharmaceutical composition comprising the compound of claim 5 and a pharmaceutically acceptable carrier.

7. The pharmaceutical composition according to claim 7, further comprising a component selected from the group consisting of: saline solution (NaCl 0.9%), Poloxamers (Pluronics), excipients, a suspension, a transporter, stabilizers and combinations thereof.

8. The pharmaceutical composition according to claim 7, wherein the pharmaceutical composition is in a topical form.

9. A method for treating cancer affecting epithelial internal tissue and epithelial external tissue the method comprising administering to a subject in need thereof an effective amount of the compound according to claim 1.

10. The compound according to claim 1 wherein said synergistic effects are selected from the group consisting of potentiating therapeutic effects, using smaller doses of anti-cancer agents, diminution of undesirable side effects, elimination of haematuria.
